(19) United States
(12) Patent Application Publication
(10) Pub. No.: US 2010/0248275 A1
(43) Pub. Date: Sep. 30, 2010

(54) METHODS FOR PREDICTING OUTCOME IN TRAUMATIC BRAIN INJURY

(75) Inventors: GEORGE JACKOWSKI, KETTLEBY (CA); ERIC B. STANTON, BURLINGTON (CA); PETRO KUPCHAK, TORONTO (CA); MIYOKO TAKAHASHI, NORTH YORK (CA); MICHELLE DAVEY, BRAMPTON (CA)

Correspondence Address: COOLEY LLP;ATTN: Patent Group, Suite 1100, 777 - 6th Street, NW, WASHINGTON, DC, 20001, US

(73) Assignee: NEXUS DX, INC., San Diego, CA (US)

(21) Appl. No.: 12/749,364

(22) Filed: Mar. 29, 2010

Related U.S. Application Data
(63) Continuation of application No. 11/737,561, filed on Apr. 19, 2007, now abandoned.
Application No. 11/737,561 is a continuation of application No. 11/201,349, filed on Aug. 10, 2005, now abandoned.
Application No. 11/201,349 is a continuation-in-part of application No. 09/940,698, filed on Aug. 27, 2001, now abandoned.

(51) Int. Cl. G01N 33/543; G01N 33/53

(52) U.S. Cl. 435/7.94; 436/518

(57) ABSTRACT

The invention describes methods for predicting outcome for patients suffering from traumatic brain injury (TBI) by evaluating levels of markers commonly associated with cellular damage in bodily fluids. Utilization of such methods improves diagnosis and treatment of patients suffering from traumatic brain injury, thus potentially minimizing and/or eliminating long-term adverse effects in these patients.

### CROSS REFERENCE TO RELATED APPLICATIONS

This application is a continuation of application Ser. No. 11/201,349, filed on Aug. 10, 2005, which is a continuation-in-part of application Ser. No. 09/940,698, filed on Aug. 27, 2001, the contents of which is herein incorporated by reference.

This application is also related to application Ser. No. 10/950,221, filed on Sep. 24, 2004, which is a continuation-in-part of application Ser. No. 09/954,972, filed on Sep. 17, 2001, the contents of both are herein incorporated by reference.

### FIELD OF THE INVENTION

The instant invention relates generally to the diagnosis and treatment of head injuries and particularly to methods for rapid assessment of subjects suffering from traumatic brain injury (TBI). The invention most particularly relates to methods for predicting outcome for subjects suffering from TBI by evaluating levels of markers commonly associated with cellular damage in bodily fluids.

### BACKGROUND OF THE INVENTION

Damage to the brain by a physical force is broadly termed traumatic brain injury (TBI). The resulting effect of TBI causes alteration of normal brain processes attributable to changes in brain structure and/or function. There are two basic types of brain injury, open head injury and closed head injury. In an open head injury, an object, such as a bullet, penetrates the skull and damages the brain tissue. Closed head injury is usually caused by a rapid movement of the head during which the brain is whipped back and forth, bouncing off the inside of the skull. Closed head injuries are the most common of the two, which often result from accidents involving motor vehicles or falls. In a closed head injury, brute force or forceful shaking injures the brain. The stress of this rapid movement pulls apart and stretches nerve fibers or axons, breaking connections between different parts of the brain. In most cases, a resulting blood clot, or hematoma, may push on the brain or around it, raising the pressure inside the head. Both open and closed head injuries can cause severe damage to the brain, resulting in the need for immediate medical attention.

Depending on the type of force that hits the head, varying injuries such as any of the following can result: jarring of the brain within the skull, concussion, skull fracture, contusion, subdural hematoma, or diffuse axonal injury. Though each person's experience is different, there are common problems that many people with TBI face. Possibilities documented include difficulty in concentrating, ineffective problem solving, short and long-term memory problems, and impaired motor or sensory skills; to the point of an inability to perform daily living skills independently such as eating, dressing or bathing. The most widely accepted concept of brain injury divides the process into primary and secondary events. Primary brain injury is considered to be more or less complete at the time of impact, while secondary injury evolves over a period of hours to days following trauma.

Primary injuries are those commonly associated with emergency situations such as auto accidents, or anything causing temporary loss of consciousness or fracturing of the skull. Contusions, or bruise-like injuries, often occur under the location of a particular impact. The shifting and rotating of the brain inside the skull after a closed brain injury results in shearing injury to the brain's long connecting nerve fibers or axons, which is referred to as diffuse axonal injury. Lacerations are defined as the tearing of frontal and temporal lobes or blood vessels caused by the brain rotating across ridges inside the skull. Hematomas, or blood clots, result when small vessels are broken by the injury. They can occur between the skull and the brain (epidural or subdural hematoma), or inside the substance of the brain itself (intracerebral hematoma). In either case, if they are sufficiently large they will compress or shift the brain, damaging sensitive structures within the brain stem. They can also raise the pressure inside the skull and eventually shut off the blood supply to the brain.

Delayed secondary injury at the cellular level has come to be recognized as a major contributor to the ultimate tissue loss that occurs after brain injury. A cascade of physiologic, vascular, and biochemical events is set in motion in injured tissue. This process involves a multitude of systems, including possible changes in neuropeptides, electrolytes such as calcium and magnesium, excitatory amino acids, arachidonic acid metabolites such as the prostagladins and leukotrienes, and the formation of oxygen free radicals.

This secondary tissue damage is at the root of most of the severe, long-term adverse effects a person with brain injury may experience. Procedures which minimize this damage can be the difference between recovery to a normal or near-normal condition, or permanent disability.

Diffuse blood vessel damage has been increasingly implicated as a major component of brain injury. The vascular response seems to be biphasic. Depending on the severity of the trauma, early changes include an initial rise in blood pressure, an early loss of the automatic regulation of cerebral blood vessels, and a transient breakdown of the blood-brain barrier (BBB). Vascular changes peak at approximately six hours post-injury but can persist for as long as six days. The clinical significance of these blood vessels changes is still unclear, but may relate to delayed brain swelling that is often seen, especially in younger people.

The process by which brain contusions produce brain nercrosis is equally complex and is also prolonged over a period of hours. Toxic processes include the release of oxygen free radicals, damage to cell membranes, opening of ion channels to an influx of calcium, release of cytokines, and metabolism of free fatty acids into highly reactive substances that may cause vascular spasm and ischemia. Free radicals are formed at some point in almost every mechanism of secondary injury. The primary target of the free radicals are the fatty acids of the cell membrane. A process known as lipid peroxidation damages neuronal, glial, and vascular cell membranes in a geometrically progressing fashion. If unchecked, lipid peroxidation spreads over the surface of the cell membrane and eventually leads to cell death. Thus, free radicals damage endothelial cells, disrupt the blood-brain barrier (BBB), and directly injure brain cells, causing edema and structural changes in neurons and glia. Disruption of the BBB is responsible for brain edema and exposure of brain cells to damaging blood-borne products.

Secondary systemic insults (outside the brain) may consequently lead to further damage to the brain. This is extremely common after brain injuries of all grades of severity, particularly if they are associated with multiple injuries. Thus, people with brain injury may experience combinations of low blood oxygen, blood pressure, heart and lung changes, fever, blood coagulation disorders, and other adverse changes at recurrent intervals in the days following brain injury. These occur at a time when the normal regulatory mechanism, by which the cerebralvascular vessels can relax to maintain an adequate supply of oxygen and blood during such adverse events, is impaired as a result of the original trauma.

The protocols for immediate assessment are limited in their efficiency and reliability and are often invasive. Computer-assisted tomographic (CT) scanning is currently accepted as the standard diagnostic procedure for evaluating TBI, as it can identify many abnormalities associated with primary brain injury, is widely available, and can be performed at a relatively low cost (Marik et al. Chest 122:688-711 2002; McAllister et al. Journal of Clinical and Experimental Neuropsychology 23:775-791 2001). However, the use of CT scanning in the diagnosis and management of patients presenting to emergency departments with TBI can vary among institutions, and CT scan results themselves may be poor predictors of neuropsychiatric outcome in TBI subjects, especially in the case of mild TBI injury (McCullagh et al. Brain Injury 15:489-497 2001).

Immediate treatment for TBI typically involves surgery to control bleeding in and around the brain, monitoring and controlling intracranial pressure, insuring adequate blood flow to the brain, and treating the body for other injuries and infection. Those with mild brain injuries often experience subtle symptoms and may defer treatment for days or even weeks. Once a patient chooses to seek medical attention, observation, neurological testing, magnetic resonance imaging (MRI), positron emission tomography (PET) scan, single-photon emission CT (SPECT) scan, monitoring the level of a neurotransmitter in spinal fluid, computed tomography (CT) scans, and X-rays may be used to determine the extent of the patient's injury. The type and severity of the injury determine further care. Unfortunately, mild brain injuries often result in long term disabilities, especially if treatment is deferred or if the patient is not followed up after treatment.

According to the Center for Disease Control, national data estimates for 1995-1996 for incidence of traumatic brain injury include the treatment and release of one million patients from hospital emergency departments, wherein for every 230,000 hospitalized who survive, 50,000 die. It is now estimated that every 15 seconds another person in the United States sustains a brain injury and that at least 5.3 million Americans are currently living with a TBI-related disability.

The cost of TBI in the United States regarding such disability, lost work wages and rehabilitation for resulting various cognitive and movement impairments total approximately 48 billion dollars, with hospitalization costs reaching 32 billion each year. This obviously does not include the human costs, or burdens borne, by those who are injured and their families.

Diagnostic techniques for the early diagnosis of traumatic brain injury and identification of the type and severity of TBI are needed to allow a physician to prescribe the appropriate therapeutic drugs at an early stage in the cerebral event and thus limit the occurrence of long-term disabilities for the patient. Various markers for brain injury are proposed and analytical techniques for the determination of such markers have been described in the art. As used herein, the term “marker” refers to a protein or other molecule that is released from the brain during a cerebral event. Such markers include isoforms of proteins that are unique to the brain.

It has been reported in the literature that various biochemical markers have correlated with cerebral events such as a traumatic brain injury. Myelin basic protein (MBP) concentration in cerebrospinal fluid (CSF) increases following sufficient damage to neuronal tissue, head trauma or AIDS dementia. Further, it has been reported that ultrastructural immunocytochemistry studies using anti-MBP antibodies have shown that MBP is localized exclusively in the myelin sheath. S-100β protein is another marker which may be useful for assessing neurological damage, for determining the extent of brain damage, and for determining the extent of brain lesions. Thus, S-100β protein has been suggested for use as an aid in the diagnosis and assessment of brain lesions and neurological damage due to brain injury, as in a stroke. Neuron specific enolase (NSE) also has been suggested as a useful marker of neurological damage in the study of brain injury, as in stroke, with particular application in the assessment of treatment. Previous studies have shown that the serum concentrations of these proteins (S-100β, NSE and MBP) correlate with the severity of TBI.

Currently, there is a clinical need for serum biochemical marker tests that can be used as an aid in the diagnosis of head injury, as potential tools in patient stratification when access to neuroimaging techniques is limited, and as prognostic aids in helping predict short-term patient outcome, especially among patients suffering from mild TBI (Quereshi AI Critical Care Medicine 30:2778-2779 2002).

If such tests can be developed and put into practice, the efficiency and quality of diagnosis and treatment options available for patients suffering from TBI would improve significantly, thus potentially minimizing and/or eliminating the occurrence of long-term adverse effects in these patients.

### PRIOR ART

Herrmann et al. (Journal of Neurotrauma 17(2):113-133 2000) aim their investigation on the release of neuronal markers (neuron specific enolase (NSE) and S-100β) and their association with intracranial pathologic changes as demonstrated by computerized tomographic (CT) scans. Their findings suggest release patterns of S-100β and NSE differ in patients with primary cortical contusions, diffuse axonal injury, and signs of cerebral edema without focal mass lesions. It is also suggested that all serum concentrations of NSE and S-100β significantly correlate with the volume of contusions. Herrmann et al. therefore suggest that NSE and S-100β may mirror different pathophysiological consequences of TBI. In a later study, Herrmann et al. (Journal of Neurology, Neurosurgery and Phychiatry 70(1):95-100 2001) examine the release patterns of neurobiochemical markers of brain damage (NSE and S-100β) in patients with traumatic brain injury and their predictive value with respect to short and long-term neuropsychological outcome. Serial NSE and S-100β concentrations are analyzed in blood samples taken at the first, second and third day after traumatic brain injury. Patients with short and long-term neuropsychological disorders are found to have significantly higher NSE and S-100β serum concentrations and a significantly longer lasting release of both markers. A comparative analysis of the predictive value of clinical, neuroradiological, and biochemical data shows initial S-100β values above 140 ng/L to have the highest predictive power. Therefore, it is suggested that the analysis of post-traumatic release patterns of neurobiochemical markers of brain damage might help to identify patients with traumatic brain injury who run a risk of long-term neuropsychological dysfunction.

Raabe et al. (Acta Neurochir. (Wein) 140(8):787-792 1998) investigate the association between the initial levels of serum S-100β protein and NSE and the severity of radiologically visible brain damage and outcome after severe head injury. Raabe et al. suggest there exists a significant correlation between different grades of diffuse axonal injury determined by Marshall classification and initial serum S-100β protein, and between the volume of contusion visible on CT scans and serum S-100β. Further, they suggest serum S-100β may provide superior information about the severity of primary brain damage after head injury.

Raabe and Seifert (Neurosurgery Review 23(3):136-138 2000) teach the use of S-100β protein independently as a serum marker of brain cell damage after severe head injury. Minor head injury is usually defined as a clinical state involving a Glasgow Coma Scale (GCS) score of 13-15; the lower the score the more severe the head injury. Patients with severe head injury (GCS≦8) are thought to be the best candidates for this study. Venous blood samples for S-100β protein are taken after admission and every 24 hours for a maximum of 10 consecutive days. Outcome is assessed at 6 months using the Glasgow Outcome Scale. Their findings indicate levels of S-100β are significantly higher in patients with unfavorable outcome compared to those with favorable outcome. In patients with favorable outcome, slightly increased initial levels of S-100β return to normal within 3 to 4 days. However, in patients with unfavorable outcome, initial levels are markedly increased, with a tendency to decrease from day 1 to day 6. After day 6, there tends to be a secondary increase in serum S-100, indicating secondary brain cell damage. Their preliminary results suggest that serum S-100β protein may be a promising biochemical marker which may provide additional information on the extent of primary injury to the brain and the prediction of outcome after severe head injury.

Rothoerl et al. (Journal of Trauma 45(4):765-767 1998) demonstrate the difference in S-100β serum levels following minor and major head injury. In minor injury, the mean serum level of S-100β within 6 hours of the injury is 0.35 μg/L. In major injury with favorable outcome, the mean serum concentration is shown as 1.2 μg/L, whereas with an unfavorable outcome the mean is 4.9 μg/L. Rothoerl et al. only identify there is a difference, but do not utilize the varying levels in the diagnosis of patients presenting with head trauma. Follow-up on the progress of patient outcome once the patient is discharged is not discussed.

Ingebrigtsen et al. (Neurosurgery 45(3):468-476 1999) are interested in the relation of serum S-100β protein measurements to MRI and neurobehavioral outcome in damage due to minor head injury. Minor head injury in this study consists of patients with a GCS score of 13-15 in whom the brain CT scans revealed no abnormalities. Serum levels are initially taken upon hospital admittance and hourly thereafter for 12 hours following injury. Analysis is by a two-site immunoradiometric assay kit. Their findings indicate a mean peak serum level of S-100β to be 0.4 pg/L in 28% of patients which were highest upon initial analysis and would decline thereafter. The patients with MRI revealing contusions also tend to have significantly higher serum S-100β levels. In addition, these patients form a trend toward impaired neuropsychological functioning on measures of attention, memory, and information processing speed, for which all patients are tested for at 3 months post-injury.

Ingebrigtsen et al. conclude that measurements of S-100β recently following head injury provide information on the extent of TBI, but most importantly also contribute early prognostic information for identification of patients on later neurobehavioral outcome, specifically, prolonged neurobehavioral dysfunction.

Fridriksson et al. (Acad. Emergency Medicine 7(7):16-820 2000) based on their findings, suggest serum NSE as a reliable marker in the prediction of intracranial lesions in children with head trauma. Their studies are based on the findings of Skogseld et al. (Acta NeuroChir. (Wein) 115:106-111 1992) and Yamazaki et al. (Surg. Neurology 43(3):267-271 1995) who suggest that serum NSE levels in patients with head trauma usually peak early after injury, reflecting the mechanical disruption of brain tissue, and then gradually fall. Although thought to be a reliable marker for predicting intracranial lesions in children, their results indicate elevated serum NSE levels in the acute phase after blunt trauma are neither sensitive nor specific in detecting all lesions. Nearly 25% of patients with intracranial lesions are missed, including patients in dire need of surgical procedures.

Yamazaki et al. (Surg. Neurology 43(3):267-271 1995) illustrates the diagnostic significance of patients with acute head injury between those who survive and those who die. Blood samples are taken following injury at a mean of 4.3 hours. Serum levels of NSE and MBP are both significantly elevated in the patients who die versus the patients who survive. For NSE, the levels are approximately 51 ng/mL versus 18 ng/mL, respectively. For MBP, the levels are approximately 11 ng/mL versus 1 ng/mL, respectively. This assay of NSE and MBP levels is suggested to provide early prediction of the prognosis on patients with acute head injury.

Myelin basic protein (MBP) is generally thought to be associated with autoimmune disease. However, MBP has also been linked with head trauma. Most significant is the study by Mao et al. (Hua Xi Yi Ke Da Xue Xue Bao; article in Chinese, 26(2):135-137 1995). Serum levels of MBP analyzed by enzyme-linked immunosorbent assay (ELISA) following acute closed head injury appear to show distinctions between type of injury. At a significantly high level of serum MBP (p<0.05) are patients with severe head injury such as cerebral contusion or intracerebral hematoma, with no significant difference between them. Much lower are patients with extradural hematoma. Patients with cerebral concussion show no significant change in serum MBP. Thomas et al. (Lancet 1(8056):113-115 1978) shows mean concentrations of MBP in patients with severe intracerebral damage, with or without extracerebral hematoma, at a significantly raised level for two weeks after injury.

U.S. Pat. No. 5,486,204 (Clifton) teaches a method for treating severe, closed head injury with hypothermia. This is done in order to diminish brain tissue loss when administered during and after ischemia. Such a method includes the administration of medications to control both the effects of the brain injury and to balance the potential deleterious effects to the body when being subjected to reduced temperatures for an extended period of time. According to the claims, a patient must be cooled for 48 hours. Not only does this method absolutely require a long period of time and proper space to perform this task, but also involves medications to combat the side effects of hypothermia, in addition to those for treating the brain injury.

Methods of assessing and treating head injuries often suggest the administration of pharmaceutical drugs as a blind test to determine the extent of the damage. This may not only be costly but also dangerous to a patient on other medications. U.S. Pat. Nos. 6,096,739; 6,090,775 and 5,527,822 all teach a method of treatment involving the administration of a pharmaceutical. U.S. Pat. No. 6,096,739 (Feuerstein) uses cytokine inhibitors, or 1,4,5-substituted imidazole compounds and compositions, to treat central nervous system (CNS) injuries to the brain. U.S. Pat. No. 6,090,775 (Rothwell et al.) uses a compound which treats the conditions of neurological degeneration by interfering with the action of interleukin-1, an agent which affects a wide variety of cells and tissues, directly modifying glial and neuronal function, and is critical in mediating inflammatory conditions. U.S. Pat. No. 5,527,822 (Scheiner) describes a method of treatment of traumatic brain injury by administering a butyrolactone derivative. This patent does describe a form of treatment based on a diagnosis of traumatic brain injury based on the presence of intracranial hypertension with direct effects on cerebral perfusion following TBI and leading to acute inflammation.

U.S. Pat. No. 6,052,619 describes the use of portable electroencephalograph (EEG) instruments to detect and amplify brain waves and convert them into digital data for analysis by comparison with data from normal groups. This is suggested for use in emergencies and brain assessments in a physician's office. Although very useful, the described invention is a medical system to transmit data, not a biochemical testing procedure.

U.S. Pat. No. 6,235,489 (Jackowski et al.) entitled “Method for Diagnosing and Distinguishing Stroke and Diagnostic Devices for Use Therein” is drawn to a method for determining whether a subject has had a stroke and, if so, the type of stroke, which includes analyzing the subject's body fluid for at least four selected markers of stroke, namely, MBP, S-100β, NSE and a brain endothelial membrane protein such as thrombomodulin or a similar molecule. The data obtained from the analyses provides information as to the type of stroke, the onset of occurrence and the extent of brain damage and allows a physician to quickly determine the type of treatment required by the subject.

The art is lacking a non-invasive point-of-care methodology useful for recent TBI sufferers to enable appropriate measures to be taken for treatment, for example, on-site in emergency situations or over a prolonged period for chronic conditions. Providing a rapid point-of-care test would enable the practitioner to quickly and definitively determine the presence of head trauma. For example, this type of test could be performed by an EMT or performed upon arrival in the ER. The importance of such a tool can be illustrated by the example of child abuse case where the infant (shaken baby syndrome) or child may not be able to express what has occurred. The proper authorities could perform the simple, inexpensive test to ensure whether abusive events have occurred and whether these events have been ongoing. In addition, the safety of the infant could be conveniently followed by intermittent testing for further signs of abuse. Another useful example lies in the sports arena. Hockey players and boxers are routinely exposed to constant forces against the head. A simple diagnostic test can determine the immediate effects of an individual concussion, or the build up of repetitive injury with each ensuing match. An acceptable level could be implemented to protect participants from dangerous levels of exposure, thus avoiding the effects of secondary injuries. Such techniques can provide data which will allow a physician to rapidly determine the appropriate treatment required by the patient and thereby permit early intervention.

Although it is well-established that measurement of biochemical markers in body fluid provides valuable information concerning the health status of a patient with a head injury, there remains a need for methods having increased sensitivity and efficiency in order to minimize and/or eliminate long-term adverse effects in these patients.

Currently, there is a clinical need for serum biochemical marker tests that can be used as an aid in the diagnosis of head injury, as potential tools in patient stratification when access to neuroimaging techniques is limited, and as prognostic aids in helping predict short-term patient outcome, especially among patients suffering from mild TBI (Quereshi AI Critical Care Medicine 30:2778-2779 2002).

If such tests can be developed and put into practice, the efficiency and quality of diagnosis and treatment options available for patients suffering from TBI would improve significantly, thus potentially minimizing and/or eliminating the occurrence of long-term adverse effects in these patients.

### SUMMARY OF THE INVENTION

The invention generally relates to methods for improving the diagnosis and treatment of head injuries in order to minimize and/or eliminate adverse effects in head trauma patients.

The S-100 protein has been highly scrutinized as a marker of brain tissue damage. Anderson et al. (Neurosurgery 48(6):1255-1260 2001) discloses that other tissues (bone, fat, muscle) also release S-100 protein after trauma, and thus, in instances wherein a patient suffers multiple traumatic injuries, interpretation of elevated S-100 levels may be difficult. The instant invention resolves these problems as documented by Anderson et al.

The present invention particularly provides a prognostic method for use in predicting poor short-term outcome for patients suffering from traumatic brain injury (TBI) by detecting elevated levels of the (3 subunit of S-100 protein in bodily fluid using an assay which is highly-specific for brain-released S-100 protein. Additionally, this assay is highly sensitive, having a detection limit of 10 pg/mL, a vast improvement over the 100 pg/mL detection limit of assays available in the prior art (Rothermundt et al. Microscopy Research and Technique 60:614-632 2003; Anderson et al. Neurosurgery 48(6):1255-1260 2001). In the exemplified experiments, the concentration of S-100 in normal control patients was undetectable, and thus, if present, was below the detection limit of the assay.

The S-100 assay used in the disclosed methods was described by Takahashi et al. (Clinical Chemistry 45(8):1307-1311 1999). This assay is highly selective for the brain specific isoform of S-100 protein. FIG. 21 illustrates prior art as reproduced from Takahashi et al. (FIG. 1A at page 1309). The data shown in this graph indicates that more of the neurological isoform S-100β was bound when carrying out the assay than the other isoforms. The solid triangle symbolizes the αα isoform; the hollow circle symbolizes the αβ isoform and the solid circle symbolizes the ββ isoform. Thus, the assay of Takahashi et al. is capable of differentiating S-100 released from the brain from S-100 released from other traumatized tissues, such as, bone, fat and/or muscle.

Conventional diagnostic methods, such as those that identify physical signs of injury, can also be applied to further improve effectiveness of the described assays. The presence of physical abnormalities in the brain is determined by imaging the brain, which may be performed using any known imaging technique, but, preferably, is performed by computer-assisted tomographic (CT) scan. Physical abnormalities of particular importance are subdural hematoma, epidural hematoma, subarachnoid hemorrhage, cerebral contusion and diffuse axonal injury.

The present invention also provides a method for predicting and/or confirming the existence of brain injury detected by a computer-assisted tomographic (CT) scan in patients suffering from traumatic brain injury (TBI) by evaluating the levels of myelin basic protein (MBP) in bodily fluids. It was found that concentrations of myelin basic protein (MBP) correlate with results of CT scans. Additionally, such an assay improves diagnosis by enabling identification of at-risk patients who otherwise might be missed using conventional diagnostic methods alone. A patient found to have an MBP level within the targeted range (elevated above 76 pg/mL) prior to undergoing a CT scan should be immediately referred for a CT scan.

According to the method, a body fluid of the patient is analyzed for at least one molecule which is cell type specific, namely, S-100β, neuron specific enolase (NSE), and myelin basic protein (MBP). The method analyzes the isoforms of the proteins which are specific to the brain tissue. The body fluid sample can be any body fluid, but is preferably blood, blood products, or cerebralspinal fluid (CSF). The biochemical markers may be utilized singly or in various combinations conclusive of various types of trauma. The analyses of these markers may be carried out on the same sample of body fluid or on multiple samples of body fluid. Different body fluid samples may be taken at the same time or at different time periods. By measuring markers in samples of body fluid taken at different periods of time, the progress of TBI can be ascertained and monitored.

The information which is obtained according to the method of the invention can be vital to the physician by assisting in the determination of how to treat a patient presenting with symptoms of TBI. The data may rule TBI in or out, and differentiate between primary and secondary TBI. The data may also determine whether there is evidence of ongoing or repetitive injury. Further, the method can provide, at an early stage, prognostic information relating to the outcome of TBI. This prognostic information can improve patient selection for appropriate therapeutics and intervention, which is especially relevant for patients diagnosed with mild TBI. Mild TBI patients often show no physical signs of injury, such as abnormalities on CT-scan, and are not always followed up, leaving these patients more vulnerable to the long-term adverse effects that may result from the TBI.

Accordingly, it is an objective of the instant invention to provide a method for predicting outcome for a subject suffering from traumatic brain injury (TBI).

It is a further objective of the instant invention to provide a method for predicting outcome for a subject suffering from traumatic brain injury (TBI) by evaluating the level of the β subunit of S-100 protein in bodily fluids.

It is a still further objective of the instant invention to provide a method for predicting and/or confirming the existence of brain injury detected by a computer-assisted tomographic (CT) scan in a subject suffering from traumatic brain injury (TBI) by evaluating the level of myelin basic protein (MBP) in bodily fluids.

It is another objective of the invention to provide such methods, which when utilized, improve diagnosis and treatment of subjects suffering from traumatic brain injury (TBI), potentially minimizing and/or eliminating long-term adverse effects in these patients.

It is a still further objective of the invention to provide prognostic kits for carrying out the methods of the instant invention.

Other objectives and advantages of the instant invention will become apparent from the following description taken in conjunction with the accompanying drawings wherein are set forth, by way of illustration and example, certain embodiments of the instant invention. The drawings constitute a part of this specification and include exemplary embodiments of the present invention and illustrate various objects and features thereof.

### BRIEF DESCRIPTION OF THE FIGURES

FIG. 1 is a data table illustrating information collected from head trauma patients

FIG. 2 shows boxplots of baseline marker levels, stratified by CT result

FIG. 3 shows boxplots of baseline marker levels in mild TBI subjects, stratified by CT result

FIG. 4 shows boxplots of baseline marker levels, stratified by outcome status two weeks post injury

FIG. 5 shows boxplots of baseline marker levels, stratified by outcome status two weeks post injury in the subset of mild TBI subjects

FIG. 6 shows a graph of time profiles for S-100β, stratified by outcome status two weeks post injury

FIG. 7 shows boxplots of S-100β levels as a function of outcome status two weeks post injury, stratified by time after injury

FIG. 8 shows a graph of time profiles for S-100β, stratified by CT result

FIG. 9 shows a graph of time profiles for MBP, stratified by CT result

FIG. 10 shows boxplots of MBP levels as a function of CT result, stratified by time after injury

FIG. 11 shows a graph of time profiles for MBP, stratified by outcome status two weeks post injury

FIG. 12 shows a graph of time profiles for NSE, stratified by outcome status two weeks post injury

FIG. 13 shows boxplots of NSE levels as a function of CT result, stratified by time after injury

FIG. 14 show a graph of time profiles for NSE, stratified by CT result

FIG. 15 shows a boxplot of S-100β levels in mild (GCS 14-15) TBI subjects, stratified by CT result and outcome status two weeks post injury

FIG. 16 shows a dotplot of baseline marker levels in TBI subjects and matching control subjects

FIG. 17 shows ROC curves assessing overall diagnostic abilities of TBI markers

FIG. 18 shows a dotplot of baseline marker levels in TBI subjects, stratified by CT result

FIG. 19 shows a dotplot of baseline marker levels in TBI subjects, stratified by short-term outcome status

FIG. 20 shows a dotplot of baseline S-100β levels, stratified by short term outcome status in the subset of mild TBI subjects

FIG. 21 shows PRIOR ART FIG. 1A as reproduced from Takahashi et al. (Clinical Chemistry 45(8):1307-1311 1999)

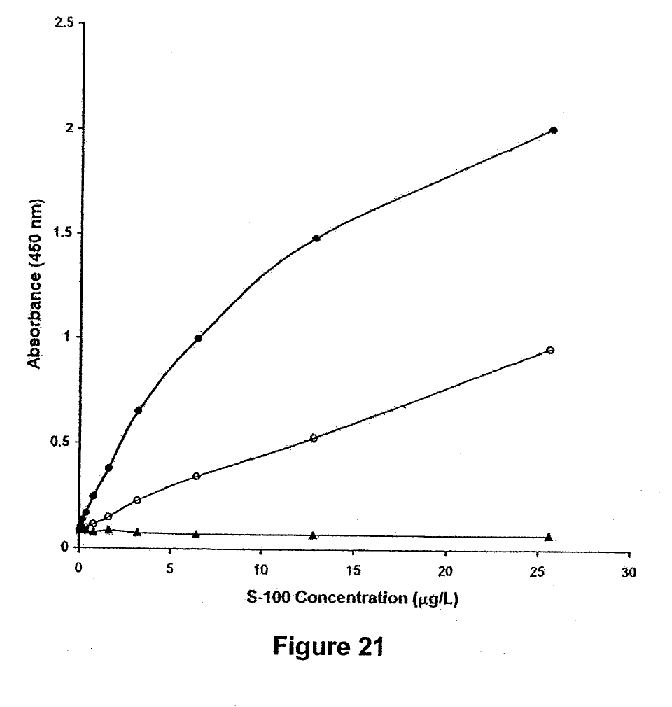

### ABBREVIATIONS AND DEFINITIONS

The following list defines terms, phrases and abbreviations used throughout the instant specification. Although the terms, phrases and abbreviations are listed in the singular tense the definitions are intended to encompass all grammatical forms.

As used herein, the term “predict” means to make known in advance, especially on the basis of special knowledge.

As used herein, the term “prognosis” is a prediction of the probable outcome and/or course of a medical condition, such as a disease or an injury.

As used herein, the term “subject” refers to an individual with symptoms of and/or suspected of traumatic brain injury. A subject is usually a human patient presenting to an emergency department. The terms “subject” and “patient” are used interchangeably herein.

As used herein, the phrase “short-term outcome” is applied to describe the health status of TBI patients included within the study described herein at 2 weeks post-TBI occurrence, determined via the telephone follow-up survey and dichotomized into good vs. poor prognosis depending on whether the TBI subject had returned to normal daily activities after 2 weeks or whether the TBI subject had not returned to normal daily activities as a direct consequence of the TBI.

As used herein, the phrase “normal daily activities” refers to the regular activities common to a person's day prior to the occurrence of the TBI.

As used herein, the phrase “long-term adverse effects” refers to prolonged impaired neuropsychological functioning that a person may experience after occurrence of TBI; including problems with attention, concentrating, short and long term memory, information processing speed, problem solving, motor skills and/or sensory skills.

As used herein, the term “sample” refers to a volume of body fluid which is obtained at one point in time.

As used herein, the abbreviation “TBI” refers to traumatic brain injury; damage to the brain caused by a physical force. Primary brain injury is considered to be more or less complete at the time of impact, while secondary injury evolves over a period of hours to days following the initial trauma. TBI is considered to be mild when a patient scores between 13 and 15 on the Glasgow Coma Scale (GCS). Mild TBI is usually associated with a loss of consciousness (LOC) for 5 minutes or less after the injury and/or amnesia for a period of 10 minutes or less after the injury. TBI is considered moderate to severe when a patient scores less than 13 on the GCS.

As used herein, the abbreviation “MVA” refers to a motor vehicle accident, involving collisions between vehicles and/or collisions between people and vehicles. Unfortunately, TBI is often a result of an MVA.

As used herein, the abbreviation “LOC” refers to the loss of consciousness that is commonly associated with TBI.

As used herein, the abbreviation “GCS” refers to the Glasgow Coma Scale; a system that is used to quantify levels of consciousness after TBI. Eye opening, verbal response and motor response are evaluated to arrive at a total score. The greater the total score the less severe the injury.

As used herein, the term “CT scan” refers to computer-assisted tomographic scanning; the current standard for evaluating TBI. CT scanning involves injection of a contrast dye followed by the use of X-rays to produce detailed pictures of the brain, from which abnormalities can be determined. In the study described herein a CT scan result was categorized as positive if evidence of at least one of the following conditions was demonstrated on the CT scan; SDH, EDH, SAH, cerebral contusion and DAI. A CT scan result was categorized as negative if the subject showed signs of skull fracture, scalp lacerations or soft tissue injury but with none of the above described conditions of brain injury.

As used herein, the term “hematoma” generally refers to bleeding within and/or around the brain.

As used herein, the abbreviation “SDH” refers to subdural hematoma; a type of TBI wherein blood collects below the inner layer of the dura but is external to the brain and arachnoid membrane (between the skull and the brain).

As used herein, the abbreviation “EDH” refers epidural hematoma; a type of TBI wherein blood collects between the inner table of the skull and the dural membrane.

As used herein, the abbreviation “SAH” refers to subarachnoid hematoma; a type of TBI wherein blood collects in the subarachnoid space.

As used herein, the term “intracerebral hematoma” refers to a type of TBI wherein bleeding occurs within the brain tissue.

As used herein, the term “cerebral contusion” refers to a bruise within the brain.

As used herein, the abbreviation “DAI” refers to diffuse axonal injury; a type of TBI wherein shearing force damages the axons and thus damages the connections between nerves within the brain. Such injury to the axons can result in a persistent vegetative state.

As used herein, the abbreviation “CSF” refers to the cerebrospinal fluid.

As used herein, the abbreviation “BBB” refers to the blood-brain barrier. The endothelial cells that make up the walls of the blood vessels in the brain are tightly packed together and, as a result, are not as permeable as the vessels in other organs. “Semi-permeable” blood vessels prevent many substances from entering the brain from the circulation. S-100β, Neuron Specific Enolase (NSE), and Myelin Basic Protein (MBP) are proteins that have been found to be elevated in the serum when the BBB has been compromised.

As used herein, the abbreviation “CNS” refers to the central nervous system (brain and the spinal cord).

As used herein, the term “marker” generally refers to any protein or other molecule which is released into the bodily fluids from injured cells and/or tissues. Particularly, with regard to the instant invention, a marker is a protein or other molecule that is released from the brain during a cerebral event. Such markers also include isoforms of proteins specific the brain. The terms “biochemical marker”, “serum marker”, “marker” and “TBI marker” are used interchangeably herein.

As used herein, the term “cerebral event” refers to any event, such as an injury, a disease process and/or infection, which results in damage to the brain cells.

As used herein, the phrase “immunologically detectable” means that a marker or a fragment of a marker contains an epitope which is specifically recognized by an antibody.

As used herein, the term “binding” refers to the ability of a protein to interact with, and form bonds with another protein.

As used herein, the phrase “specifically binding” refers to the ability of an antibody to specifically interact with and form bonds with an epitope of a particular antigen.

As used herein, the term “S-100β” refers to a cytoplasmic, acidic, calcium-binding protein. The protein exists in several homodimeric or heterodimeric isoforms consisting of two immunologically distinct subunits, alpha α (MW=10,400 Dalton) and beta β (MW=10,500 Dalton). The isoform S-100 β, is the 21,000 Dalton homodimer ββ and is found primarily in neurological cells (astrocytes and Schwann cells). The isoform S-100α is the heterodimer αβ which is also found in neurological cells. The isoform S-100 αα is the homodimer found mainly in striated muscle, heart and kidney (Isobe et al. European Journal of Biochemistry 115:469-474 1981; Isobe et al. Journal of Neurochemistry 43:1494-1496 1984; Semba et al. Brain Research 401:9-13 1987; Kato et al. Biochem. Biophys. Acta 842:146-150 1985). The assay of the instant invention is specific for the β subunit of the S-100 protein, and it measures the 13 subunit concentration in both the ββ and αβ isoforms of the protein.

As used herein, the abbreviation “NSE” refers to neuron specific enolase, a glycolytic enzyme found in neurons and neuroendocrine cells.

As used herein, the abbreviation “MBP” refers to myelin basic protein, a protein found in growing oligodendroglial cells and is bound to the extracellular membranes of central and peripheral myelin (myelin sheath).

As used herein, the abbreviation “ELISA” refers to an enyzme-linked immunosorbent assay or “sandwich” assay. In an ELISA assay antibody or antigen is coated onto a solid phase and an enzyme reaction is used to detect and quantify the substance of interest.

As used herein, the term “ROC curve” refers to a receiver operating characteristic curve which is used to interpret the value of diagnostic tests. For example, the number of patients with and without a disease is graphed to produce the curves. There is an area of overlap in the patient number distributions as no diagnostic test can be 100% effective. The area of overlap defines where the test is ineffective for detecting the disease. In practice a cutoff line is determined above which the test is considered abnormal and below which the test is considered normal. The accuracy of the test is determined by how well the test discriminates patients without the disease from patients with the disease. Accuracy is determined by calculating the area under the curve (AUC).

As used herein, the terms “above normal” and “above threshold” refer to a level of a marker that is greater than the level of the marker observed in normal individuals, that is, individuals who are not undergoing a cerebral event (an injury to the brain which may be ischemic, mechanical or infectious). Frequently, diagnostic and/or prognostic information can be gleaned from marker concentrations elevated above a normal cut-off range. For some markers, no or infinitesimally low levels of the marker may be present normally in an individual's blood. For others of the markers analyzed, detectable levels may be present normally in blood. Thus, these terms contemplate a level that is significantly above the normal level found in individuals. The term “significantly” refers to statistical significance and generally means a two standard deviation (SD) above normal, or higher concentration of the marker is present. The assay method by which the analysis for any particular marker protein is carried out must be sufficiently sensitive to be able to detect the level of the marker which is present over the concentration range of interest and also must be highly specific.

### DETAILED DESCRIPTION OF THE INVENTION

Serum levels of markers commonly associated with cellular damage in the brain are evaluated in this invention to predict outcome for patients suffering from traumatic brain injury. The markers which are analyzed are released into circulation following injury and are present in the blood and other body fluids. Preferably blood, or any blood product such as, for example, plasma, serum cytolyzed blood (e.g., by treatment with hypotonic buffer or detergents), and dilutions and preparations thereof are analyzed according to the invention. In another embodiment the concentration of markers in cerebrospinal fluid (CSF) is measured.

The primary markers which are measured according to the present method are proteins which are released by the specific brain cells as the cells become damaged during a cerebral event. These proteins can either be in their native form or immunologically detectable fragments of the proteins resulting, for example, by enzyme activity from proteolytic breakdown.

The markers analyzed according to the method of the invention are cell type specific. Myelin basic protein (MBP) is a highly basic protein, localized in the myelin sheath, and accounts for about 30% of the total protein of the myelin in the human brain. The protein exists as a single polypeptide chain of 170 amino acid residues which has a rod-like structure with dimensions of 1.5×150 nm and a molecular weight of about 18,500 daltons. It is a flexible protein which exists in a random coil devoid of α helices and β conformations.

The increase of MBP concentration in blood and CSF in cerebral hemorrhage is highest almost immediately after the onset. A normal value for a person who has not had a cerebral event is from 0.00 to about 0.016 ng/ml. MBP has a half-life in serum of about one hour and is a sensitive marker for cerebral hemorrhage.

The S-100 protein is a cytoplasmic acidic calcium binding protein found predominantly in the grey matter of the brain, primarily in the glia and Schwann cells. The protein exists in several homo- or heterodimeric isoforms consisting of two immunologically distinct subunits, alpha (MW=10, 400 daltons) and beta (MW=10,500 dalton). The S-100α is the homodimer αα which is found mainly in striated muscle, heart and kidney. The S-100β isoform is the 21,000 dalton homodimer ββ. It is present in high concentration in glial cells and Schwann cells and is thus brain tissue specific. It is released during acute damage to the central nervous system (CNS) and is a sensitive marker for cerebral infarction. It is eliminated by the kidney and has a half-life of about two hours in human serum. Repeated measurements of S-100β serum levels are useful to follow the course of neurologic damage. The S-100 assay disclosed in the instant invention is specific for the β subunit of the S-100 protein.

The enzyme enolase (EC 4.2. 1.11) catalyzes the interconversion of 2-phosphoglycerate and phosphoenolpyruvate in the glycolytic pathway. The enzyme exists in three isoproteins, each the product of a separate gene. The gene loci has been designated ENO1, ENO2 and ENO3. The gene product of ENOL is the non-neuronal enolase (NNE or a), which is widely distributed in various mammalian tissues. The gene product of ENO2 is the muscle specific enolase (MSE or α), which is localized mainly in the cardiac and striated muscle, while the product of the ENO3 gene is the neuron specific enolase (NSE or γ), which is largely found in neurons and neuroendocrine cells. The native enzymes are found as homo- or heterodimeric isoforms composed of three immunologically distinct subunits, α β and γ. Each subunit has a molecular weight of approximately 39,000 daltons.

The α, αγ and γγ enolase isoforms, which have been designated NSE each have a molecular weight of approximately 80,000 daltons. It has been shown that NSE concentration in CSF increases after experimental focal ischemia and the release of NSE from damaged cerebral tissue into the CSF reflects the development and size of the infarcts. NSE has a serum half-life of about 48 hours and its peak concentration has been shown to occur later after cerebral artery (MCA) occlusion. NSE levels in CSF have been found to be elevated in acute and/or extensive disorders including subarachnoid hemorrhage and acute cerebral infarction.

The data obtained according to the method indicates whether a traumatic brain injury has occurred, and, if so, the type of injury, primary or secondary. Where all markers analyzed are negative, i.e., within the normal range, there is no indication of TBI. When the level of any marker analyzed is at least 2SD above the normal range, there is indication of trauma. Depending on which markers and the degree of marker level, severity can be determined. Prior art data have indicated that possible conclusions to be drawn are very high MBP and S-100β are indicative of contusion or intracerebral hematoma; high S-100β but normal after 3-4 days indicates a favorable outcome; high S-100β for 1-6 days and then up again, indicates an unfavorable outcome; high MBP for 2 weeks indicates an unfavorable outcome and raised S-100β with no raise in MBP is indicative of a concussion.

As a result of the study described herein it has been determined that a level of S-100β elevated above 39 pg/mL indicates that a patient is likely to suffer adverse effects as a result of their injury. Likewise, a level of MBP elevated above 76 pg/ml predicts and/or confirms the existence of a brain injury as was detected by computer-assisted tomographic (CT) scan.

According to another preferred embodiment, a fourth marker, which is from the group of axonal, glial and neuronal markers analyzed according to the method of this invention, is measured to provide information related to the time of onset of the TBI. It should be recognized that the onset of TBI symptoms is not always known, particularly if the patient is unconscious or elderly. Additionally, a reliable clinical history is not always available. An indication of the time of onset of TBI can be obtained by relying on the release kinetics of brain markers of different molecular weights. The time release of brain markers into the circulation following brain injury is dependent on the size of the marker, with smaller markers tending to be released earlier in the event, while larger markers tend to be released later.

As stated previously, the level of each of the specific markers in the patient's body fluid can be measured from one single sample or one or more individual markers can be measured in one sample and at least one marker measured in one or more additional samples. By “sample” is meant a volume of body fluid which is obtained at one point in time. Further, all of the markers can be measured with one assay device or by using a separate assay device for each marker in which aliquots of the same body fluid sample can be used or different body fluid samples can be used. It is apparent that the analyses should be carried out within some short time frame after the sample is taken, e.g., within about a half hour, so the data can be used to decide treatment as quickly as possible. It is preferred to measure each of the markers in the same single sample, irrespective of whether the analyses are carried out in a single analytical device or in separate such devices so that the level of each marker simultaneously present in a single sample can be used to provide meaningful data.

Generally speaking, the presence of each marker is determined using antibodies specific for each of the markers and detecting immunospecific binding of each antibody to its respective cognate marker. Any suitable immunoassay method may be utilized, including those which are commercially available, to determine the level of each of the specific markers measured according to the invention. Extensive discussion of the known immunoassay techniques is not required here since these techniques are known to those of skill in the art. Typical suitable immunoassay techniques include sandwich immunoassays (ELISA), radio immunoassays (RIA), competitive binding assays, homogeneous assays, heterogeneous assays, etc. Various known immunoassay methods are reviewed in Methods in Enzymology, 70, pages 30-70 and 166-198; 1980. Direct and indirect labels can be used in immunoassays. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include: radionuclides and fluorescent or luminescent moieties. Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease. For a detailed discussion of enzymes in immunoassays, see Engvall, Enzyme Immunoassay ELISA and EMIT, Methods of Enzymology, 70, pages 419-439; 1980.

A preferred immunoassay method for use according to the invention is a double antibody technique for measuring the level of marker proteins in the patient's body fluid. According to this method, one of the antibodies is a “capture” antibody and the other antibody is a “detector” antibody. The capture antibody is immobilized on a solid support which may be of any of the various types which are known in the art such as, for example, microtiter plate wells, beads, tubes and porous materials such as nylon, glass fibers and other polymeric materials. In this method, a solid support, e.g., microtiter plate wells, coated with a capture antibody, preferably monoclonal, raised against the particular marker of interest, constitutes the solid phase. Diluted patient body fluid, e.g., serum or plasma, typically about 25 μl, standards and controls are added to separate solid supports and incubated. When the marker protein is present in the body fluid it is captured by the immobilized antibody which is specific for the protein. After incubation and washing, an anti-McHale marker protein detector antibody, e.g., a polyclonal rabbit anti-marker protein antibody, is added to the solid support. The detector antibody binds to marker protein bound to the capture antibody to form a sandwich structure. After incubation and washing and anti-IgG antibody, e.g., a polyclonal goat anti-rabbit IgG antibody labeled with an enzyme such as horseradish peroxidase is added to the solid support. After incubation and washing, a substrate for the enzyme is added to the solid support followed by incubation and the addition of an acid solution to stop the enzymatic reaction. The degree of enzymatic activity of immobilized enzyme is determined by measuring the optical density of the oxidized enzymatic product on the solid support at the appropriate wavelength, e.g. 450 nm for horseradish peroxidase. The absorbance at the wavelength is proportional to the amount of marker protein in the fluid sample. A set of marker protein standards is used to prepare a standard curve of absorbance vs. marker protein concentration. This immunoassay is preferred since test results can be provided in 45 to 50 minutes and the method is both sensitive over the concentration range of interest for each marker and is highly specific.

The assay methods used to measure the marker proteins should exhibit sufficient sensitivity to be able to measure each protein over a concentration range from normal value found in healthy persons to elevated levels, for example, 2 standard deviations (SD) above normal and beyond. Of course, a normal value range of the marker proteins can be found by a analyzing the body fluid of healthy persons. For the S-100β isoform where +2SD=0.02 ng/mL the upper limit of the assay range is preferably about 5.0 ng/mL. For NSE where +2SD=9.9 ng/mL the upper limit of the assay range is preferably about 60 ng/mL. For MBP, which has an elevated level cutoff value of 0.02 ng/mL, the upper level limit of the assay range is preferably about 5.0 ng/mL.

The assays can be carried out in various assay device formats including those described in U.S. Pat. Nos. 4,906,439; 5,051,237 and 5,147,609 to PB Diagnostic Systems, Inc.

The assay device used according to the invention can be arranged to provide a semi-quantitative or quantitative result. By the term “semi-quantitative” is meant the ability to discriminate between a level which is above the elevated marker protein value, and a level which is not above that threshold.

The assays may be carried out in various formats including, as discussed previously, a microtiter plate format which is preferred for carrying out the assays in a batch mode. The assays may also be carried out in automated immunoassay analyzers which are well known in the art and which can carry out assays on a number of different samples. These automated analyzers include continuous/random access types. Examples of such systems are described in U.S. Pat. Nos. 5,207,987 and 5,518,688 to PB Diagnostics Systems, Inc. Various automated analyzers that are commercially available include the OPUS and OPUS MAGNUM analyzers. Another assay format which can be used according to the invention is a rapid manual test which can be administered at the point-of-care at any location. Typically, such point-of-care assay devices will provide a result which is above or below a threshold value, i.e., a semi-quantitative result as described previously.

Furthermore, the presence of physical abnormalities in the brain is determined by imaging the brain of the patient. Imaging may be performed by any imaging technique known in the art, but is preferably performed by computer-assisted tomographic (CT) scan. The presence of any physical abnormality is noted. Abnormalities of particular interest are subdural hematoma, epidural hematoma, subarachnoid hemorrhage, cerebral contusion and diffuse axonal injury.

The level of myelin basic protein (MBP) in a sample can be evaluated in order to predict and/or confirm the presence of the brain injury, if such an injury has been detected by a CT scan. Elevated MBP levels can also be used to determine which patients should be referred for CT scans. Additionally, by evaluating marker levels and imaging results, the physician can estimate the amount of time that will pass before the patient returns to normal daily activities after the occurrence of the TBI.

### Example

A blinded case-controlled study was undertaken to compare serum levels of S-100β, NSE, and MBP in patients with TBI to age- and sex-matched control patients without TBI. The study was also useful for determining whether there is a correlation of serum levels of S-100β, NSE, and MBP with neurological findings and for determining if there is a correlation of serum levels of S-100β, NSE, and MBP with short-term functional outcome status at 2 weeks post-TBI.

### Blinded Case-Control Study

Serum levels of S-100β, neuron specific enolase (NSE) and myelin basis protein (MBP) were measured in patients presenting to the emergency department of a major urban trauma center with symptoms consistent with traumatic brain injury (TBI) and compared with serum levels of these proteins in non-TBI subjects who were matched in age and gender.

Traumatic brain injury (TBI) results in the release of biochemical markers into the bloodstream in sufficient quantities such that the serum concentrations of these markers in TBI subjects may be elevated with respect to those in age and gender matched control subjects without TBI. Serum marker concentrations may also be elevated in TBI subjects with acute brain abnormalites due to the injury, as evident on the initial CT scan, with respect to those TBI subjects with no visible abnormalities. Serum marker concentrations may also be elevated in TBI subjects with poor-short term functional outcomes, with respect to TBI subjects with good short-term outcomes.

This study was a single-center blinded case-control experiment. A total of 50 TBI subjects and 50 age and gender matched non-TBI control subjects were included in the study. The study was conducted at the emergency department of Sunnybrook and Women's College Health Sciences Center in Toronto, Ontario between September 2001 and December 2002. Approval of the study was obtained by the hospital's research ethics board prior to commencement of the study, and patients or their legally authorized representatives were required to sign Informed Consent forms prior to inclusion in the study. Both male and female subjects were included in the study who were at least 16 years of age. In order to be included as TBI subjects, patients must have presented to the emergency department within 6 hours of the initial injury, and have had an initial GCS score of 14 or less, or a GCS score of 15 with witnessed loss of consciousness (LOC) or amnesia. Patients with a known history of neurological disease, neuropsychiatric disorders or malignant melanomas were excluded from the study, as were subjects undergoing brain or spinal cord surgery within one month prior to the injury. A patient who presented to the emergency department with a condition unrelated to head trauma, with a GCS score of 15 and no witnessed LOC or amnesia, of the same gender, and with an age at enrollment within 3 years of an enrolled TBI subject, was enrolled as a matching non-TBI control subject.

Serum samples were collected from all enrolled subjects during the baseline evaluation. Serum samples were frozen at −80° C. and shipped on dry ice to SYN-X Pharma Inc. (Toronto, Ontario) for subsequent evaluation of marker levels. S-100β levels were determined using an enzyme-linked immunosorbent assay (ELISA) with a monoclonal anti-S-100β capture antibody and a polyclonal rabbit anti-S-100β detector antibody (Takahashi et al. Clinical Chemistry 45:1307-1311 1999). NSE levels were determined using an ELISA with a monoclonal anti-NSE capture antibody and a monoclonal anti-NSE detector antibody. MBP levels were determined using an ELISA with a goat polyclonal anti-MBP capture antibody and a monoclonal anti-MBP detector antibody. The detection limits for the respective assays were 10 pg/mL for S-100β, 1 ng/mL for NSE and 20 pg/mL for MBP. SYN-X Pharma personnel running the assays were blinded as to the identity of the subgroup (TBI vs. control) to which individual samples belonged.

CT scan reports were made available to the primary investigator for the subset of TBI subjects for whom CT scans were clinically indicated by the attending physician. Enrolled TBI subjects were contacted by phone approximately 2 weeks following the injury for the purpose of follow-up evaluation, using the Canadian CT Head and Cervical Spine Radiography Study Telephone Follow-Up survey. This assessment tool has been previously validated in a large clinical study of mild TBI subjects (Steill et al. Lancet 357:1391-1396 2001).

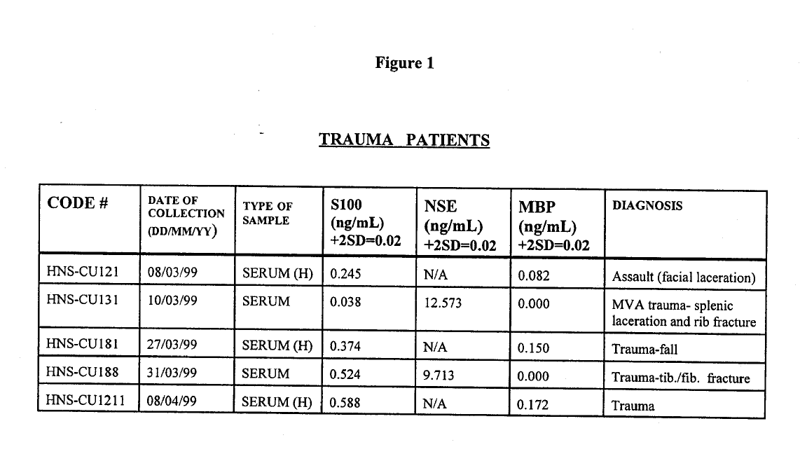

FIG. 1 displays an example of a table charting information collected from head trauma patients. Data was collected on paper case report forms by the research personnel at the investigative site. To ensure quality data for analysis, the data was verified against the source documents, entered in the clinical databases (Microsoft Access 2000 and <<SyMetric>>) and reviewed. Logical and integrity checks were performed, and all generated queries were resolved by the site. All data management procedures were conducted according to Good Clinical Practice and standards established by SYN-X Pharma.

The primary outcome measures were the serum concentrations of biomarkers as determined from the baseline blood sample drawn from each enrolled subject. A secondary outcome measure with respect to the subset of TBI subjects was the presence of a visible abnormality as determined from the initial CT scan. In particular, subjects were classified as CT-positive if evidence of at least on of the following was demonstrated on the CT-scan: subdural hematoma (SDH), epidural hematoma (EDH); subarachnoid hemorrhage (SAH), cerebral contusion and diffuse axonal injury (DAI). Subjects with signs of non-depressed skull fracture, scalp lacerations or soft tissue injury but with none of the above signs of brain injury were classified as CT-negative.

Another secondary outcome measure with respect to the subset of TBI subjects was short-term prognosis, determined via the telephone follow-up survey and dichotomized into good vs. poor prognosis depending on whether the TBI subject had returned to normal daily activities after 2 weeks or whether the subject had not returned to normal daily activities as a direct consequence of the TBI.

Summary statistics for baseline marker levels were computed with respect to both TBI subjects and non-TBI control subjects. For each biomarker, comparisons between TBI and control groups were made using Wilcoxon rank-sum tests. Receiver operating characteristic (ROC) curves were generated, and areas under the curve (AUC) were computed to provide a basis of comparison for each of the markers to discriminate between TBI subjects and non-TBI control subjects. An optimal cutoff (defined in terms of the largest sum of sensitivity and specificity) was identified from the ROC curve for each marker. Pairwise comparisons of AUC values between markers were conducted following the procedure of Hanley and McNeil (Radiology 148:839-843 1983).

With respect to the subgroup of TBI subjects, summary statistics for baseline marker levels were computed for both CT-positive and CT-negative subjects. For each biomarker, comparisons between CT-positive and CT-negative subjects were made using Wilcoxon rank-sum tests. ROC curves were generated and AUC's and optimal cutoffs for each of the markers was computed to determine the relative abilities of the biomarkers to discriminate between CT-positive and CT-negative subjects. For markers which correlated with CT result, the above analyses was repeated with respect to the subgroup of mild (GCS 14-15) TBI subjects. Biomarker levels were dichotomized using the optimal ROC cutoffs, and the baseline severity of TBI was dichotomized to GCS≦13 vs. GCS 14-15; logistic regression analyses were conducted using these dichotomized variables to determine whether biomarker levels predicted the occurrence of abnormalities on the CT scan after adjusting for the severity of TBI.

With respect to the subgroup of TBI subjects, summary statistics for baseline marker levels were computed for subjects with good short-term prognoses and for those with poor short-term prognoses. For each biomarker, comparisons between TBI subjects with good vs. poor short-term prognoses were made using Wilcoxon rank-sum tests; ROC curves were generated and AUC's and optimal cutoffs were computed. For markers which correlated with short-term prognosis, the above analyses were repeated with respect to the subgroup of mild (GCS 14-15) TBI subjects and with respect to the subgroup of mild CT-negative TBI subjects. Biomarker levels were dichotomized using the optimal ROC cutoffs, CT scan results were dichotomized to the presence vs. absence of abnormalities on the CT scan, and the baseline severity of TBI was dichotomized to GCS 3-13 vs. GCS 14-15; logistic regression analyses were conducted using these dichotomized variables to determine whether biomarker levels predicted outcome status after adjusting for the occurrence of abnormalities on the CT scan and for the severity of TBI.

ROC curve analyses were performed using MedCalc Version 7.1 (MedCalc Software, Mariakerke, Belgium); all other statistical analyses were conducted using S-Plus Version 6 for Windows (Insightful Corporation, Seattle, Wash.).

### Results

### Characteristics of Study Subjects

Blood samples were not available for one TBI subject, who died shortly after admission. Of the 49 remaining subjects, (65%) were male. A total of 34 (69%) of the TBI subjects were Caucasian, 3 were Black, 7 were Asian and 5 were of other races. The median age range of TBI subjects was 42, with a range of 16 to 89. A total of 27 (55%) of the TBI subjects had baseline GCS scores of 14 or 15 (with 22 of these being GCS 15), and 22 had baseline GCS scores of 13 or less. The majority of the injuries were motor vehicle related, with 21 (41%) occurring to drivers or passengers in vehicles involved in collisions, and another 11 (22%) occurring to pedestrians or cyclists struck by motor vehicles. Of the remaining injuries, 12 (24%) were caused by falls of various types, 2 were caused by industrial accidents, one was sports-related, one was the result of an assault, and one was caused by a flying object.

Out of the 21 drivers or passengers involved in motor vehicle collisions, 12 (57%) suffered from severe TBI (as evidenced by a baseline GCS score of 13 or less); in comparison, 11 out of 27 (41%) of subjects with other mechanisms of injury suffered from severe TBI. There was a significant association between gender and severity of TBI, as 56% of males suffered from severe TBI as opposed to only 24% of females (Fisher's exact p=0.038; 95% confidence interval for true difference in proportions between males and females=[6%. 59%]). There was no significant association between gender and mechanism of injury or between gender and age among TBI subjects. There was no significant association between race and gender, between race and age, between race and mechanism of injury or between race and severity of injury among TBI subjects.

### Baseline Marker Levels

In 2 cases, the amount of serum obtained from the TBI subject was deemed insufficient for testing of biomarkers, and in 2 other case, sample hemolysis compromised the NSE result: therefore, baseline levels of all three TBI markers were obtained for 45 of the 49 matched pairs. Table 1 displays summary statistics for baseline levels of all three markers in TBI and matching control subjects. Concentrations are given in ng/mL for NSE and in pg/mL for MBP and S-100β.

TABLE 1

| | TBI (n = 45) | | | | | Control (n = 45) | | | | |
|---|---|---|---|---|---|---|---|---|---|---|
| Marker | Min | Q1 | Median | Q3 | Max | Min | Q1 | Median | Q3 | Max |
| S-100β | 0 | 12 | 48 | 159 | 421 | 0 | 0 | 0 | 0 | 55 |
| NSE | 3.2 | 6.9 | 12.5 | 19.5 | 85 | 2.4 | 3.5 | 4.6 | 7.3 | 39 |
| MBP | 40 | 60 | 76 | 146 | 2010 | 0 | 49 | 60 | 82 | 336 |

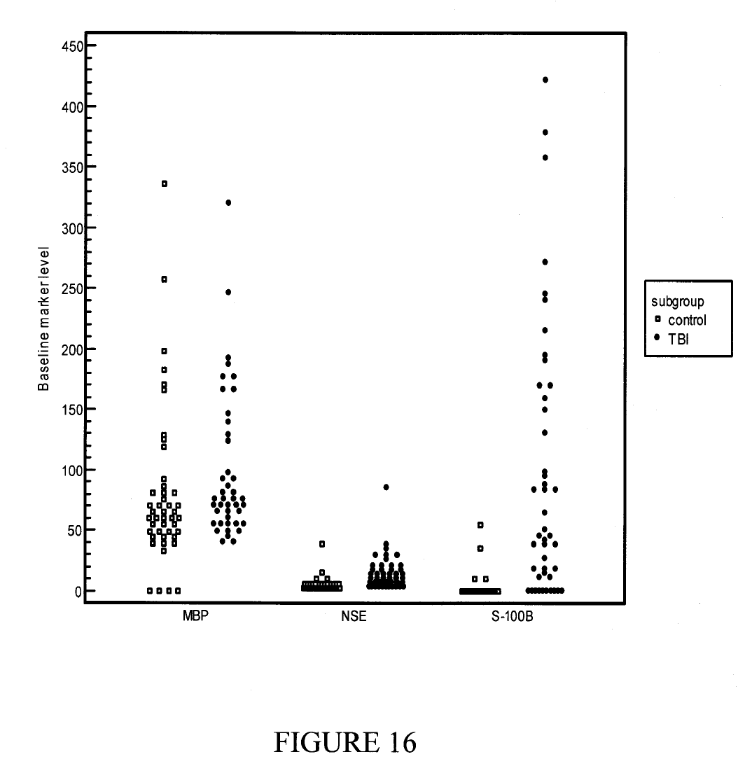

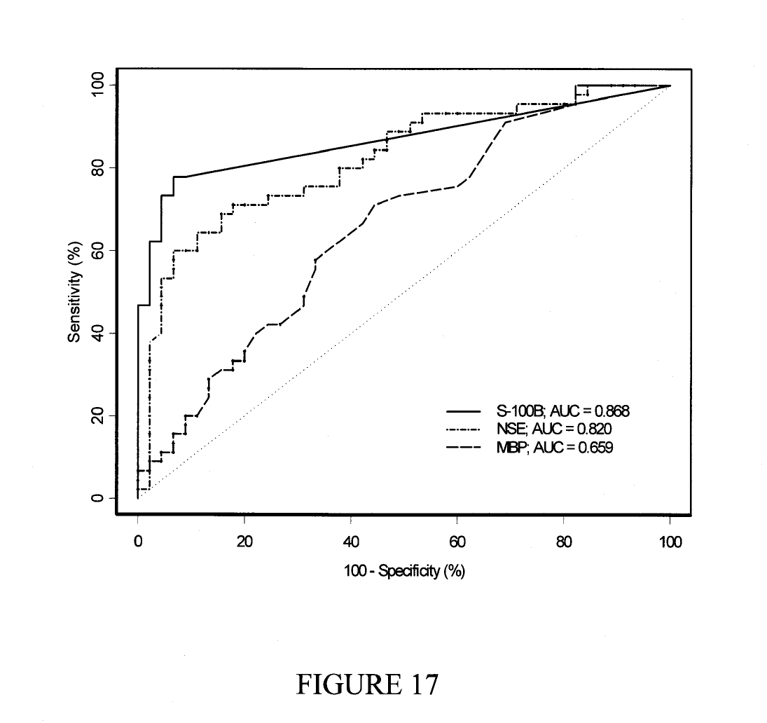

FIG. 16 displays dotplots of baseline marker levels in TBI and matching control subjects. Concentrations are given in ng/mL for NSE and in pg/mL for MBP and S-100β. Data in FIG. 16 was stratified by subgroup; TBI (symbol, solid dot) vs. control (symbol, square). S-100β showed the highest specificity of the markers, with 42 of the 45 control samples (93%) having S-100β levels at or below 10 pg/mL, the detection limit of the assay; conversely, 35 of 45 TBI subjects (78%) had detectable levels of serum S-100. TBI subjects had a median NSE level of 12.5 ng/mL (interquartile range=[6.9, 19.5]), whereas control subjects had a median NSE level of 4.6 ng/mL (interquartile range=[3.5, 7.3]). TBI subjects had a median MBP level of 76 pg/mL (interquartile range=[60, 146], whereas control subjects had a median MBP level of 60 pg/mL (interquartile range=[49, 82]). Wilcoxon tests revealed that S-100β (p<0.001), NSE (p<0.001) and MBP (p=0.009) levels were significantly higher in TBI subjects than in control subjects. FIG. 17 displays receiver operator characteristic (ROC) curves for each of the three markers, S-100β displayed the highest overall ability to discriminate between TBI and control subjects, with an area under the curve (AUC) of 0.868, as compared with an AUC of 0.820 for NSE and 0.659 for MBP. Pairwise comparisons revealed that both S-100 (p=0.001) and NSE (p=0.018) showed significantly higher discriminatory ability than MBP in this respect, whereas the discriminatory ability of S-100β was not significantly higher than that of NSE.

TABLE 2

| | | | | Sensitivity at | Specificity at |
|---|---|---|---|---|---|
| Marker | AUC | Optimal cutoff | | cutoff | cutoff |
| S-100β | 0.868 | 10 | pg/mL | 77.8% | 93.3% |
| NSE | 0.820 | 8.15 | ng/mL | 71.1% | 82.2% |
| MBP | 0.659 | 65 | pg/mL | 71.1% | 55.6% |

Table 2 summarizes the overall ROC curve analyses and shows optimal cutoffs and sensitivity and specificity estimates associated with these cutoffs.

Correlation with CT Scan Results

CT scans were performed on 39 of the 45 TBI subjects for whom baseline levels of all three markers were available. All TBI subjects with baseline GCS scores of less than 15 obtained CT scans; the 6 who were discharged without having a CT scan performed (5 falls and 1 assault victim) all experienced witnessed loss of consciousness for a period of 5 minutes or less and/or a period of post-traumatic amnesia for a period of 10 minutes or less. Of the 39 subjects undergoing CT scans, 21 of the subjects were classified as CT-positive and 18 were classified as CT-negative. The following frequencies were observed with respect to specific abnormalities as detected on the CT scan: 11 subjects with skull fracture, 10 SDH, 15 SAH, 14 with cerebral contusions, 2 with EDH and 1 with DAI. There was a significant association between severity of TBI and CT result; 15 out of 19 subjects (79%) with a baseline GCS score of 13 or less had abnormalities on the initial CT scan, in comparison with 6 out of 20 subjects (30%) with a baseline GCS score of 14 or 15 having associated abnormalities on the initial CT scan (Fisher's exact p=0.004; 95% confidence interval for true difference in proportions between severe and mild TBI subjects=[22%, 76%]).

TABLE 3

| | CT-positive (n = 21) | | | | | CT-negative (n-18) | | | | |
|---|---|---|---|---|---|---|---|---|---|---|
| Marker | Min | Q1 | Median | Q3 | Max | Min | Q1 | Median | Q3 | Max |
| S-100β | 0 | 21 | 83 | 170 | 421 | 0 | 38 | 74 | 167 | 245 |
| NSE | 4.9 | 8.5 | 13.7 | 21 | 38 | 3.3 | 6.5 | 13.5 | 21 | 85 |
| MBP | 51 | 76 | 97 | 177 | 2010 | 40 | 52 | 67 | 76 | 246 |

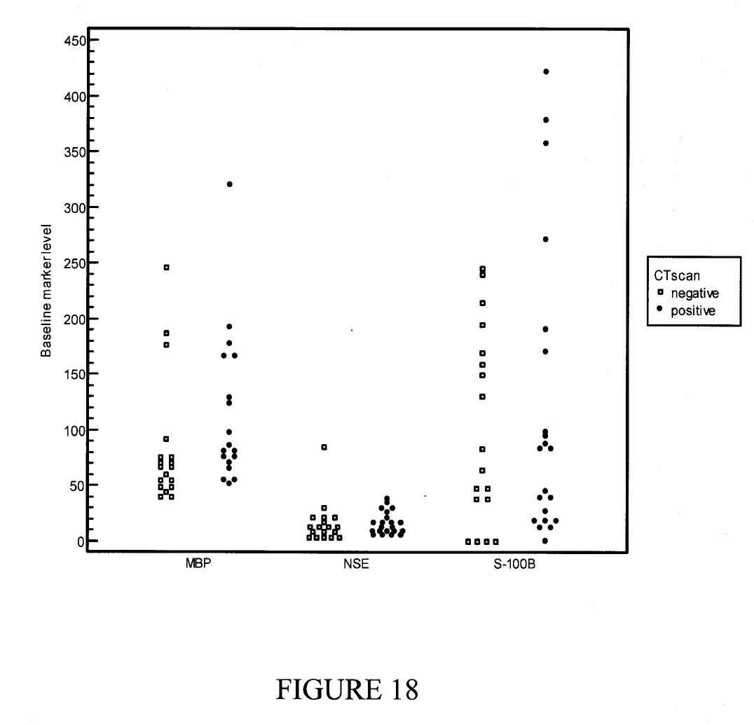

Table 3 shows a summary of the statistics with respect to baseline marker levels in TBI subjects, stratified by CT result (concentrations are given in ng/mL for NSE and in pg/mL for MBP and S-100β). FIG. 18 shows dotplots of baseline marker levels in TBI subjects, stratified by CT result (concentrations are given in ng/mL for NSE and in pg/mL for MBP and S-100β; symbols; solid dot is CT-positive and square is CT-negative). Of the three markers, MBP provided the best discrimination between CT-positive and CT-negative cases. CT-positive subjects had a median MBP level of 97 pg/mL (interquartile range=[76,177]), whereas control subjects had a median MBP level of 67 pg/mL (interquartile range=[52, 76]). Wilcoxon rank-sum tests revealed that MBP levels were higher in CT-positive subjects than in CT-negative subjects (p=0.007), but S-100β (p=0.921) and NSE (p=0.632) could not discriminate between CT-positive and CT-negative subjects. When using CT result as the classification variable, ROC analyses showed that the AUC was 0.754 for MBP, 0.546 for NSE and 0.511 for S-100β. At a cutoff of 76 pg/mL, MBP had a sensitivity of 71% (15/21) and a specificity of 78% (14/18) in distinguishing between CT-positive and CT-negative subjects. Logistic regression analyses revealed that a baseline MBP level greater than 76 pg/mL remained a significant predictor of CT result after adjusting for severity of TBI (p=0.003); with respect to the subset of mild TBI subjects alone, median baseline MBP levels showed a more than a two fold increase when comparing CT-positive and CT-negative subjects (148 pg/mL vs. 69 pg/mL). MBP also proved to be a robust predictor of individual injury patterns on the CT scan, with an AUC of greater than 0.7 in predicting the presence of each SDH, SAH and cerebral contusion.

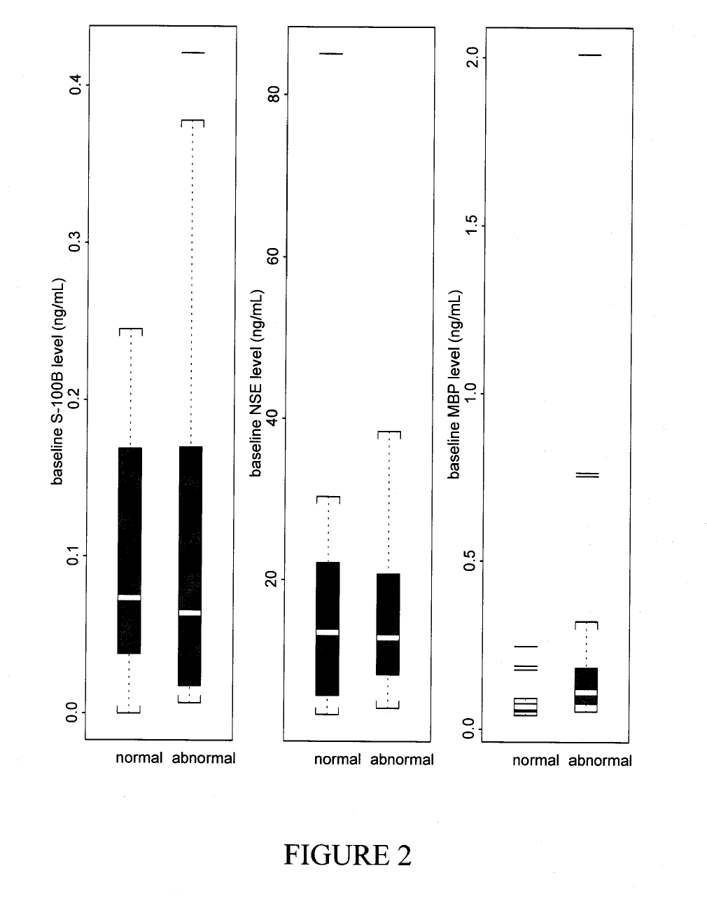

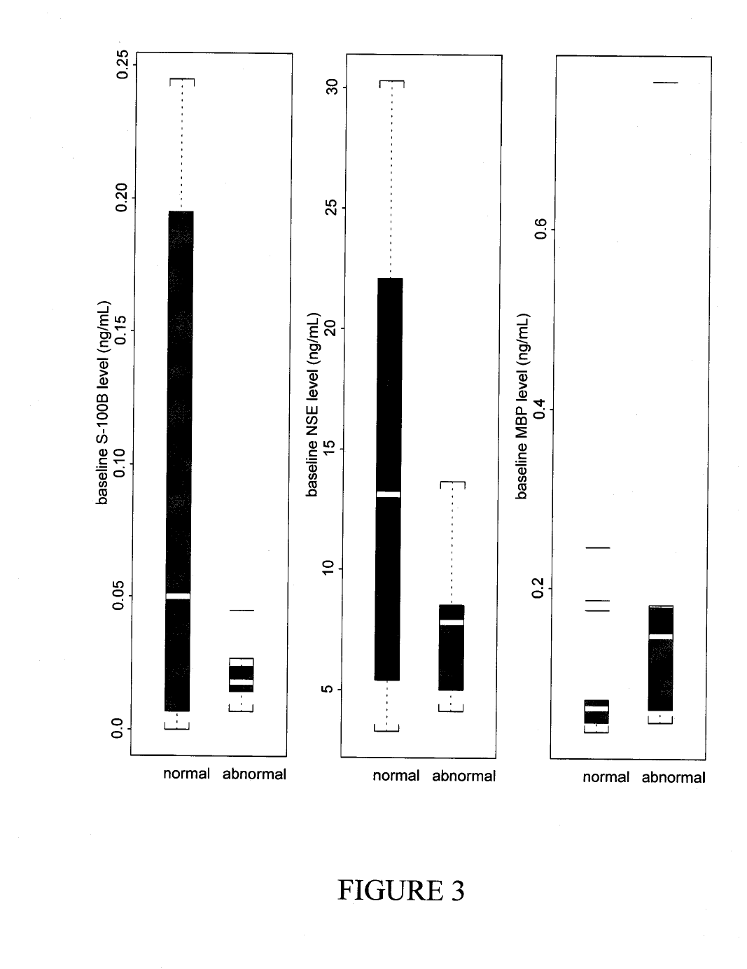

A subset of 40 TBI patients was also analyzed with respect to correlation of CT scan results. All of these patients had baseline levels of MBP, NSE and S-100β available. 23 of these patients were mild TBI cases and 17 were moderate or severe cases. 22 of these patients were classified as CT-positive and 18 were CT-negative. FIG. 2 shows boxplots of baseline marker levels, stratified by CT result. CT-positive patients had significantly higher MBP levels (p=0.008, Wilcoxon signed rank test), whereas S-100β and NSE levels did not differ significantly between CT-positive and CT-negative patients. There was a correlation of between baseline marker levels and CT scan results with respect to mild TBI subjects; S-100β and NSE levels appear to be higher in mild TBI subjects who turn out to be CT-negative, whereas MBP levels are higher in mild TBI subjects who are CT-positive. FIG. 3 shows boxplots of baseline marker levels in mild TBI subjects, stratified by CT result. Logistic regression analyses suggests that MBP is a significant predictor of CT abnormalities (p=0.005), and that neither S-100β nor NSE are significant predictors after adjusting for baseline MBP level (p=0.833 and 0.712, respectively). A subject's baseline GCS score is a significant predictor of CT abnormalities (p=0.005); after adjusting for baseline GCS score, MBP remains a significant independent predictor of CT abnormalities (p=0.007). After adjusting for baseline GCS score and baseline MBP level, NSE is also shown to be a significant predictor of CT abnormalities (p=0.043), in the sense that lower NSE levels are correlated with higher probabilities of positive CT scan results.

Correlation with Outcome Status after Two Weeks

Of the TBI subjects who were discharged from the emergency department, a total of 29 were followed up after a two week period and asked (via telephone survey) various questions concerning their health status. Short-term outcomes (good prognosis vs. poor prognosis) were classified according to whether or not the patient had returned to normal daily activities two weeks post-TBI. Ten of the 29 subjects reported having returned to normal daily activities after 2 weeks, whereas 19 had not returned to normal daily activities as a direct result of their injury (indicating poor prognosis).

TABLE 4

| | Back to normal activities | | | | | Not back to normal activities | | | | |
|---|---|---|---|---|---|---|---|---|---|---|
| | (n = 10) | | | | | (n = 19) | | | | |
| Marker | Min | Q1 | Median | Q3 | Max | Min | Q1 | Median | Q3 | Max |
| S-100β | 0 | 2 | 12.5 | 25.5 | 50 | 0 | 16.5 | 48 | 154 | 357 |
| NSE | 3.8 | 4.4 | 6.4 | 12.2 | 17.2 | 3.2 | 7 | 13.8 | 20 | 34 |
| MBP | 55 | 73 | 108 | 169 | 187 | 40 | 55 | 70 | 124 | 765 |

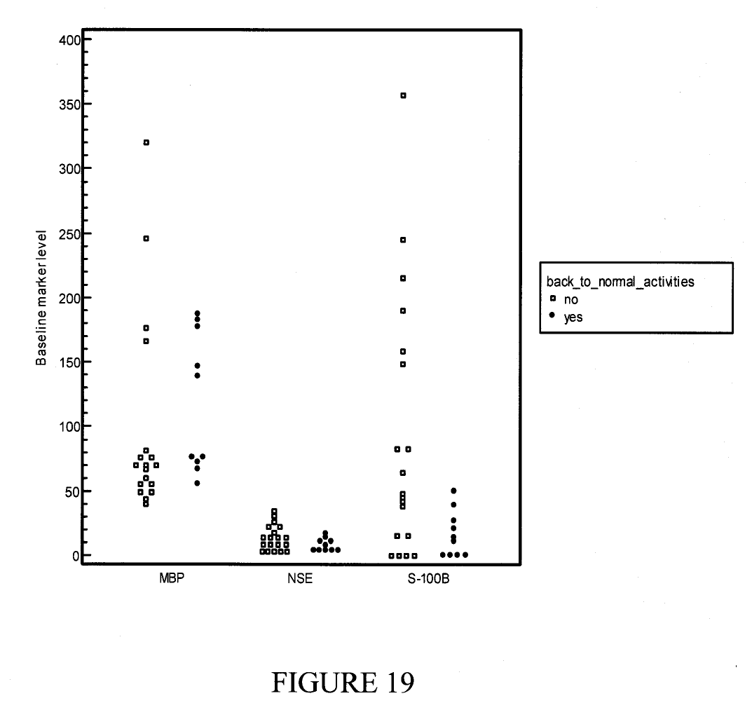

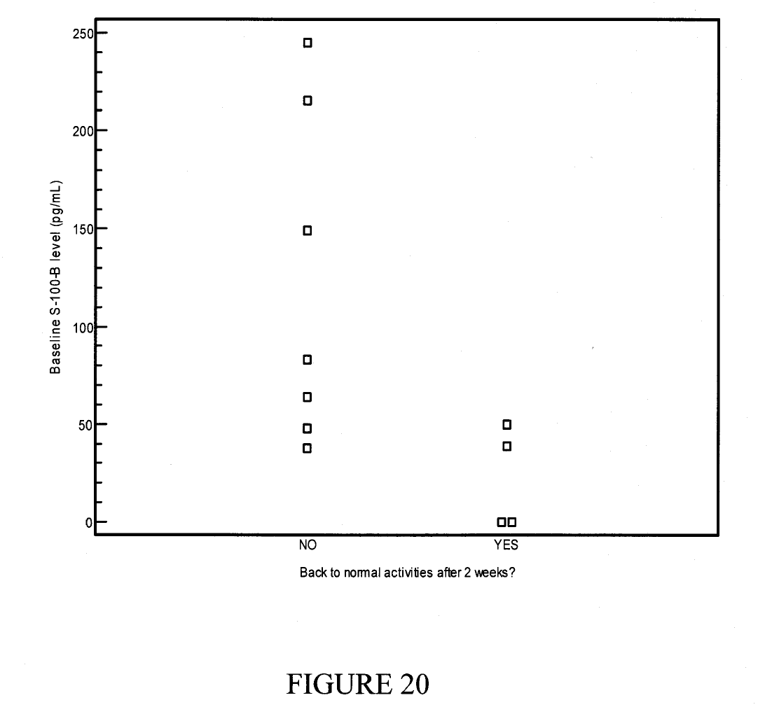

Table 4 displays summary statistics for baseline marker levels, stratified by short-term outcome. (concentrations are given in ng/mL for NSE and in pg/mL for MBP and S-100β). FIG. 19 displays dotplots of baseline marker levels stratified by short-term outcome. (concentrations are given in ng/mL for NSE and in pg/mL for MBP and S-100β; the solid dot symbol represents a return to normal daily activities and the square symbol represents that the patient has not returned to normal daily activities). S-100β provided the best discrimination between subjects with good short-term prognosis (returning to normal activities after 2 weeks) and those with poor short-term prognosis. Subjects with a poor prognosis had a median S-100β level of 48 pg/mL (interquartile range=[16.5, 154]), whereas subjects with a good prognosis had a median S-100β level of 12.5 ng/mL (interquartile range=[2,25.5]). The difference was statistically significant (Wilcoxon rank-sum p=0.022). Subjects with a poor prognosis had a median NSE level of 18.8 ng/mL (interquartile range=[7,20]), whereas subjects with a good prognosis had a median NSE level of 6.4 ng/mL (interquartile range=[4.4,12.2]); the difference was not statistically significant (p=0.069). MBP (p=0.183) could not discriminate between TBI subjects with good vs. poor outcomes. When using short-term prognosis as the classification variable, ROC analyses showed that the AUC was 0.763 for 5-100β, 0.711 for NSE and 0.655 for MBP. The optimal cutoff for S-100β, as identified in the ROC analysis using outcome status as the classification variable, was 39 pg/mL; 9 of 16 TBI subjects with baseline S-100β below this cutoff (56%) were back to normal daily activities within 2 weeks, as opposed to only 1 of 13 subjects (8%) with baseline S-100β levels above this cutoff (Fisher's exact p=0.008; 95% confidence interval for true difference in proportions=[20%. 77%]). For 24 of the TBI subjects, both CT scan results and follow-up date on short-term outcome were available; 17 of these were mild TBI subjects. CT results did not predict outcome status in this particular subset of subjects, with 3 out 11 CT-positive subjects (27%) returning to normal daily activities after two weeks, as opposed to 4 out of 13 (31%) CT-negative subjects. Logistic regression analyses revealed that baseline GCS severity predicted outcome status (p=0.015), but CT results did not (p=0.851). After adjusting for severity of TBI and CT result, a baseline S-100β level of greater than 39 pg/mL predicted outcome status (p=0.018); NSE and MBP were not significant predictors of outcome status after adjusting for severity of TBI and CT result. Within the subset of mild TBI subjects with negative CT scans (n=11; 4 with good outcomes, 7 with poor outcomes), baseline S-100β level remained a significant predictor of poor outcome (Wilcoxon rank-sum p=0.047). Baseline S-100β levels are elevated in mild TBI subjects with a poor outcome status after 2 weeks, irrespective of whether the subject was CT-positive or CT-negative. FIG. 20 displays a dotblot of baseline S-100β levels, stratified by short-term outcome status, with respect to the subset of mild CT-negative TBI subjects.

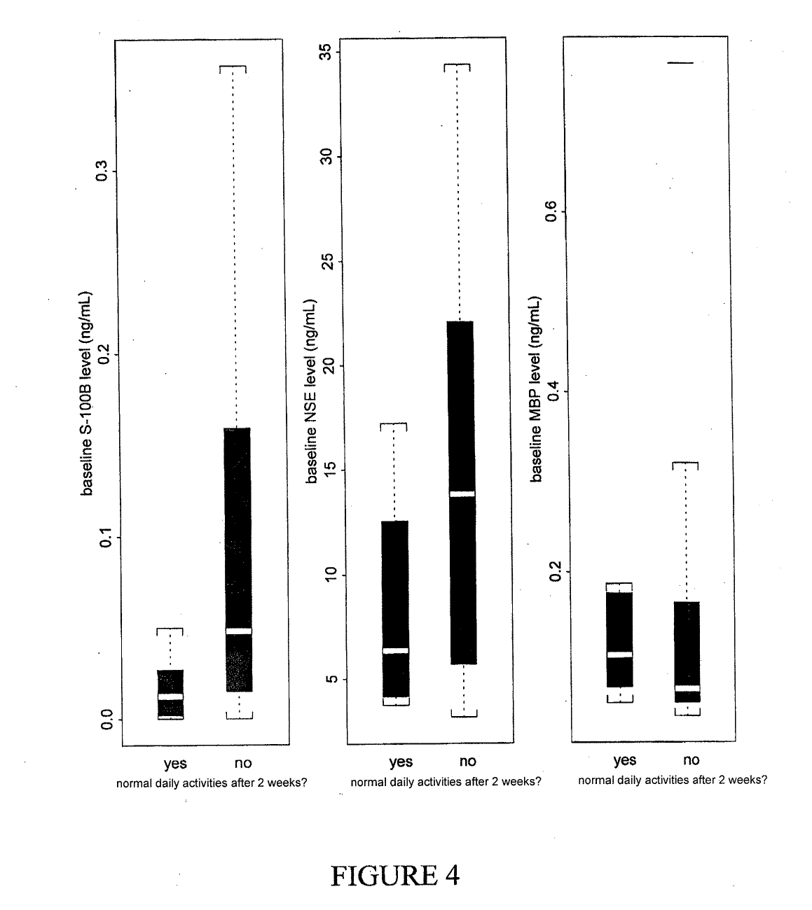

FIG. 4 displays boxplots of baseline marker levels stratified by outcome status after 2-weeks (with regard to the 29 TBI patients who were followed-up). High baseline S-100β levels predicted negative outcomes (p=0.019, Wilcoxon rank sum test), whereas NSE was a marginally significant predictor (p=0.069) and MBP was not a significant predictor in this respect (p=0.183).

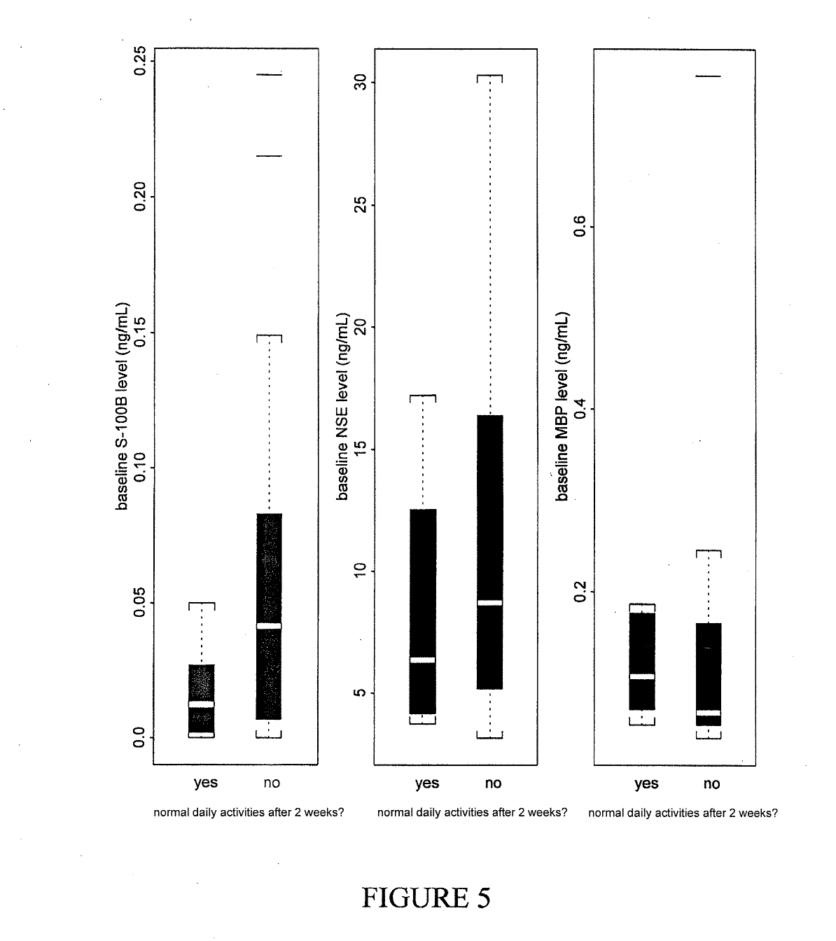

FIG. 5 displays boxplots of baseline marker levels stratified by outcome status after 2 weeks, with respect to the subset of mild TBI subjects. When examining the subset of 24 subjects who were mild TBI cases (GCS 13-15) and for whom 2-week follow-up data was available, it was found that S-100 and NSE levels are elevated in subjects who had not returned to normal daily activities after 2 weeks (FIG. 5). The differences were not found to be statistically significant in this respect (p=0.112 for S-100β and p=0.259 for NSE). Logistic regression analyses suggested that S-100β is a significant predictor of outcome status after 2 weeks (p=0.028) and that NSE is a marginally significant predictor (p=0.068); NSE does not remain significant after adjusting for S-100β (p=0.811). A subject's baseline GCS score is a significant individual predictor of outcome status after 2 weeks (p=0.010); after adjusting for baseline GCS score, S-100β remains a marginally significant predictor of outcome status (p=0.078), while NSE does not retain its significance as an independent predictor (p=0.189). After adjusting for both baseline GCS score and CT result, S-100β remains a significant individual predictor of 2 week outcome status (p=0.047).

A subset of 49 patients was also analyzed with respect to correlation of mild TBI, marker levels and outcome status.

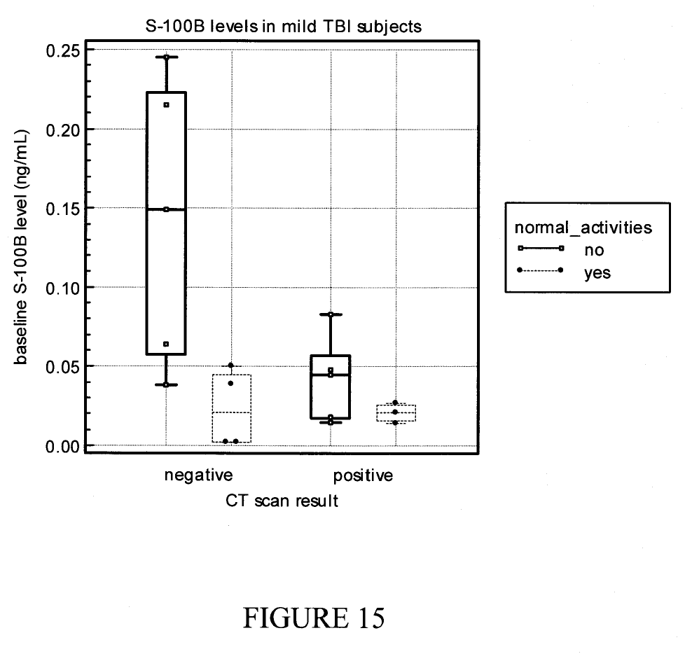

Of the 49 TBI patients for whom marker levels were available, 22 were considered as moderate or severe TBI and 27 were considered as mild TBI. CT scans were performed on 43 of these 49 patients; the 6 subjects who did not receive CT scans were all GCS 15 subjects who underwent relatively short periods of amnesia and/or LOC. CT scan results and 2-week outcome status reports were available for 17 of the mild TBI subjects. Of the subgroup of 7 subjects who were back to normal daily activities after two weeks, 3 or 43% had positive CT scans. Of the subgroup of 10 subjects who were not back to normal daily activities after 2 weeks, only 5 or 50% were CT positive. Thus, CT results were not correlated with 2 week outcome status in the subgroup of mild TBI subjects. When applying logistic regression analyses to this subset of mild TBI subjects, and after adjusting for TBI outcome, S-100β remained a significant predictor of 2 week outcome status (p=0.003), with NSE having a weak correlation with outcome status (p=0.099). MBP was not correlated with outcome status in mild TBI subjects after adjusting for CT result (p=0.906). Five of these 17 subjects were CT negative yet were not back to daily normal activities after 2 weeks, and S-100β levels were positive in all 5 of these subjects (all 5 with baseline S-100β levels of at least 0.038 ng/mL). Of the 4 CT negative subjects who were back to normal daily activities after 2 weeks, 2 had positive S-100β levels and 2 had negative levels (0.002 ng/mL). FIG. 15 displays S-100β levels in mild TBI subjects, stratified by CT result and outcome status. For mild CT-negative TBI patients who would otherwise be discharged from the emergency room, a positive S-100β baseline level could be a flag for the attending physician to ensure that the patient is followed up more closely, perhaps via an outpatient clinic.

Correlation of Marker Levels at Multiple Time Points with Outcome and CT Scan Results

As previously mentioned, a total of 29 TBI subjects were followed up after a 2 week period and asked (via telephone survey) various questions concerning their health status. Ten of the 29 subjects reported having returned to normal daily activities after 2 weeks, whereas 19 had not returned to normal daily activities as a direct result of their injury.

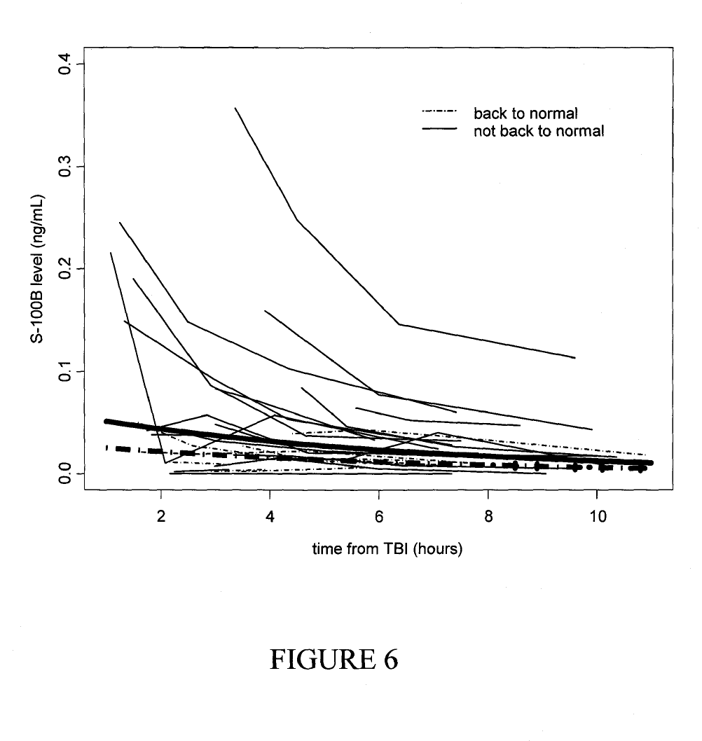

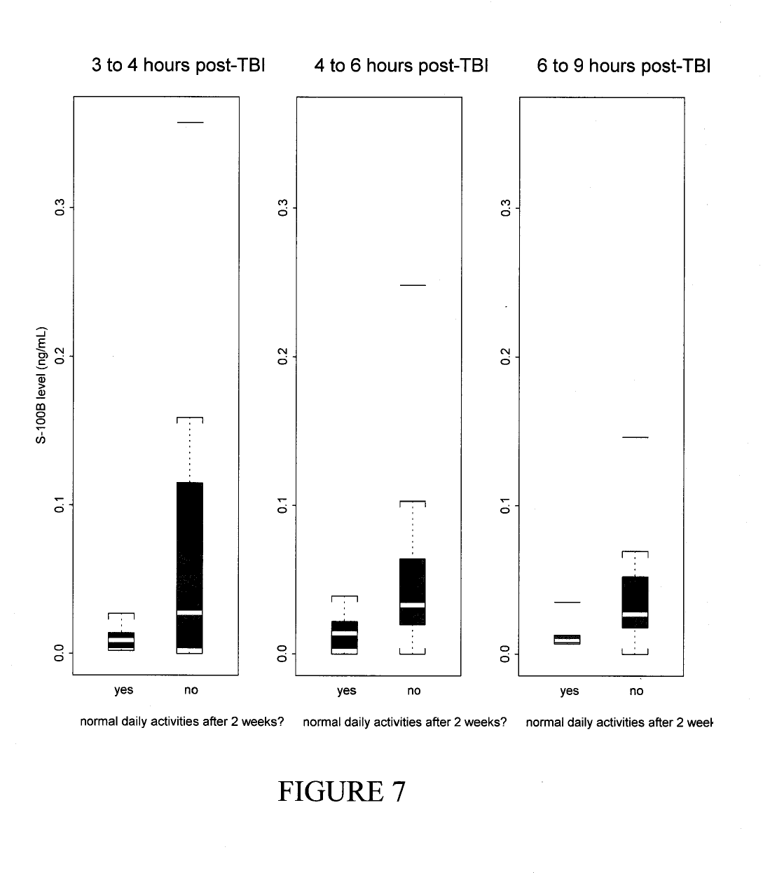

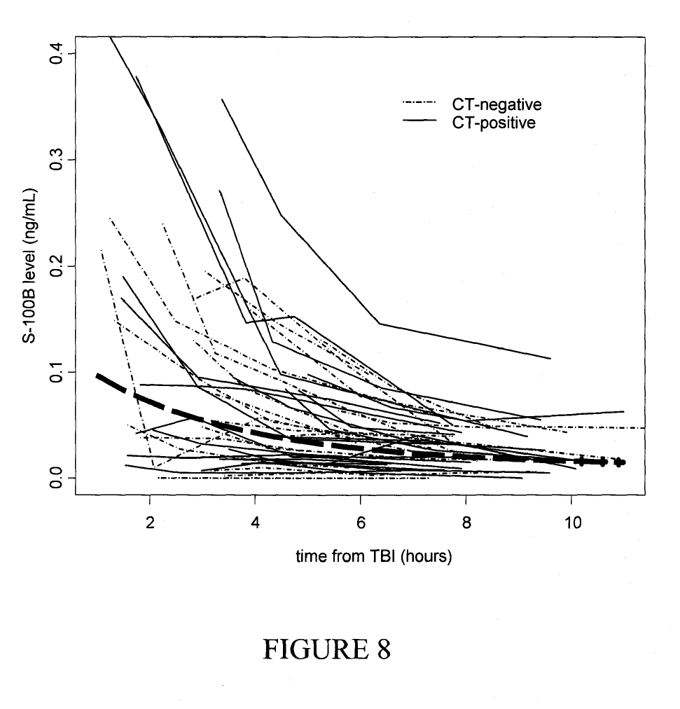

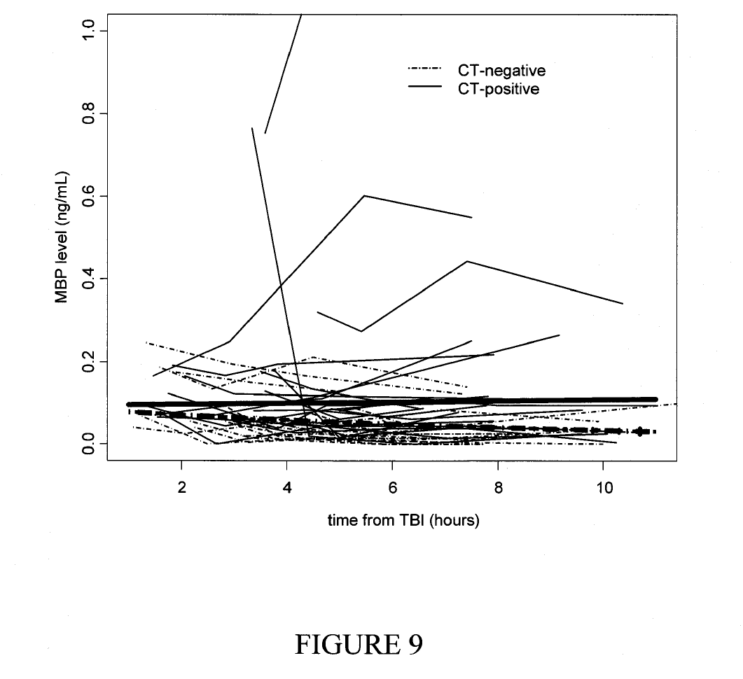

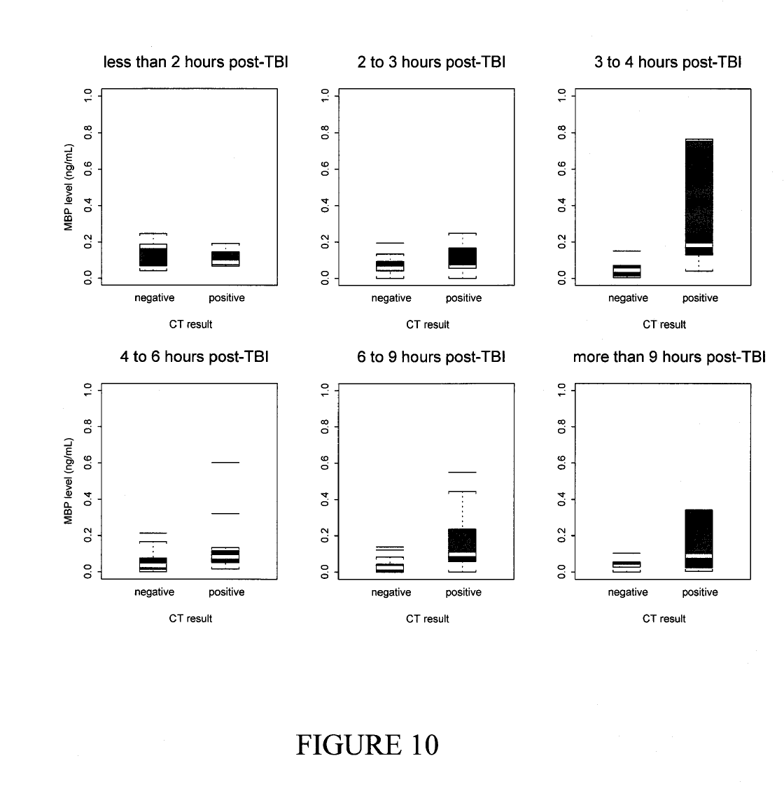

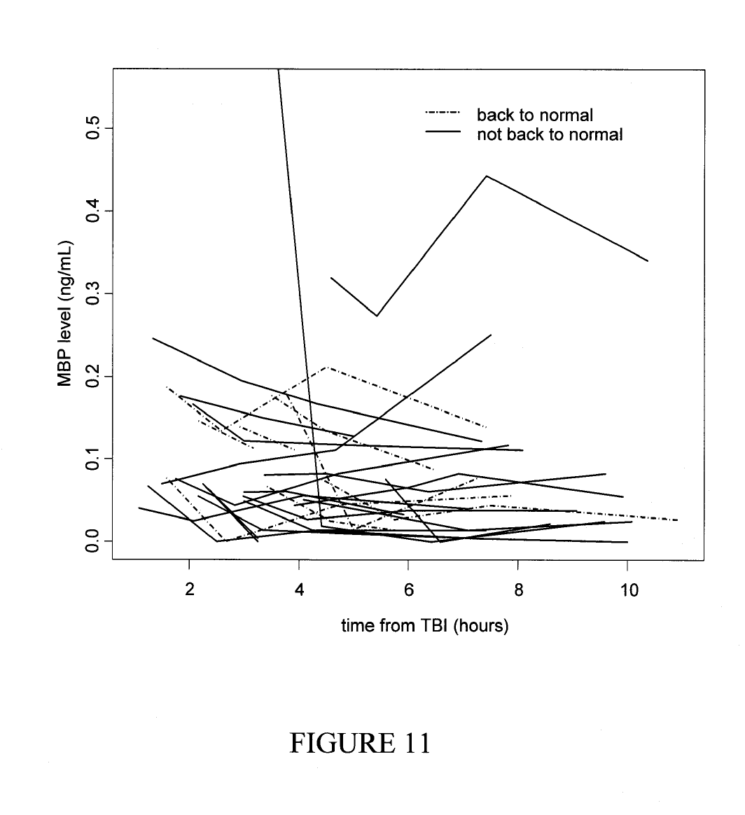

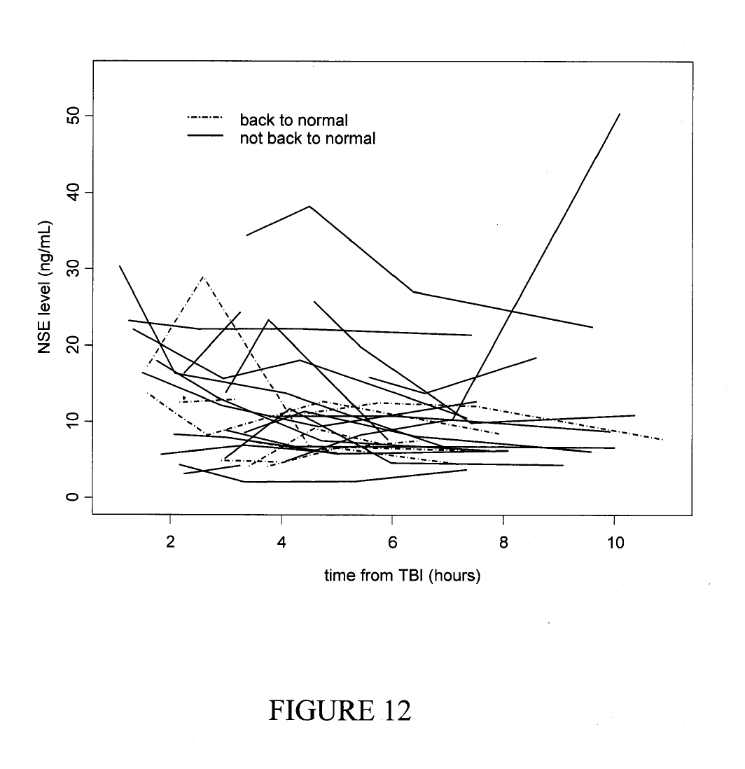

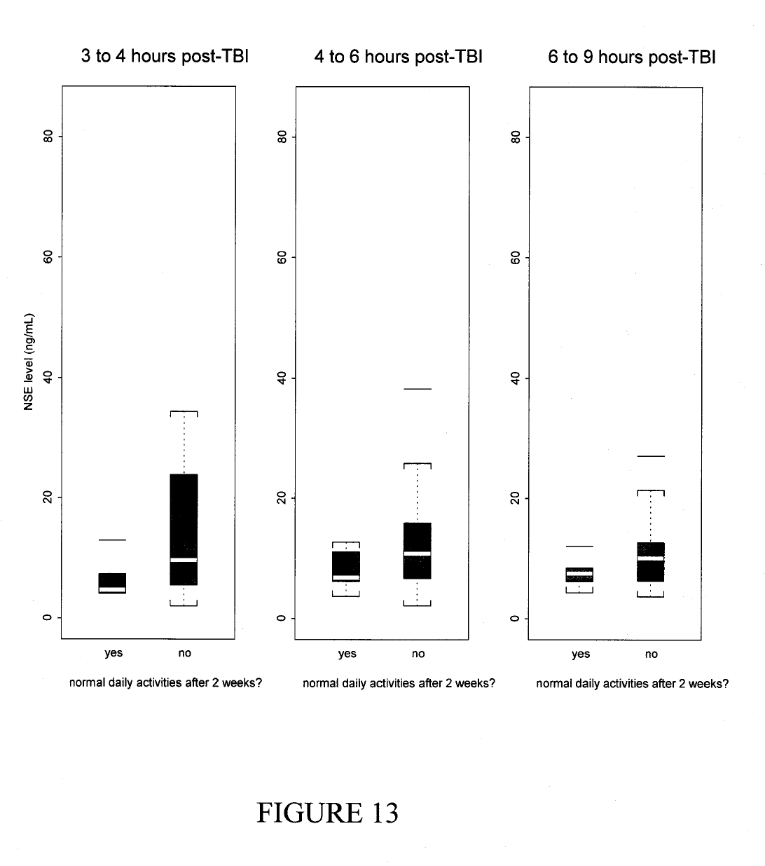

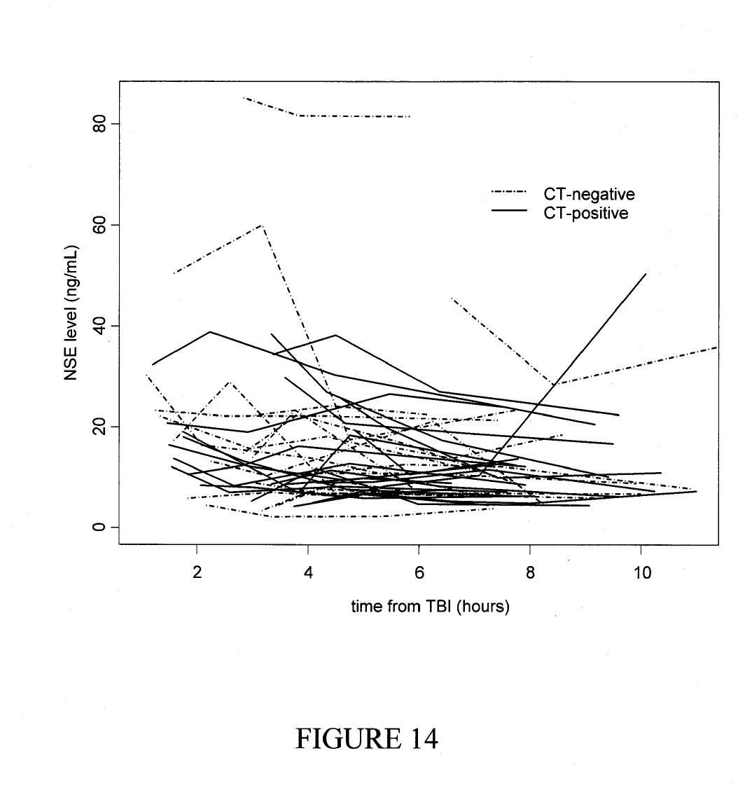

FIG. 6 displays time profiles of S-100β levels, both in subjects who had returned to normal daily activities after 2 weeks and in subjects who were not back to normal. Mixed model analyses revealed that the mean fitted curve for S-100β as a function of time after TBI is higher for subjects with poor outcomes; this difference is marginally statistically significant (p=0,086, likelihood ratio test). The S-100β time profiles in FIG. 6 were stratified by 2-week outcome status. The thick solid line and thick dotted line represent fitted models for 100β vs. time from TBI for subjects with poor outcome and subjects with good outcomes, respectively. NSE levels as a function of time after TBI are also higher in subjects with poor outcomes (p=0.111). FIG. 12 displays NSE time profiles, stratified by 2-week outcome status. FIG. 7 displays boxplots of S-100β levels in subjects with good vs. poor outcomes, stratified by categories of time after TBI. The separation between subjects with good vs. poor outcomes is most marked in a time period of 4-6 hours post-TBI (p=0.016, Wilcoxon rank sum test), and there is still separation evident 6 to 9 hours post-TBI (p=0.078). FIG. 13 displays boxplots of NSE levels as a function of 2-week outcome status, stratified by time after injury. There was no significant separation between subjects with good vs. poor outcomes in terms of NSE levels at any of the categories of time post-TBI. FIG. 11 displays time profiles for MBP, stratified by 2-week outcome status. Time profiles of MBP levels in subjects with poor outcomes do not differ in a statistical sense from those in subjects with good outcomes. CT scan results were also correlated with time profiles. A patient was classified as CT-positive if evidence of at least one of the following showed up on the CT scan; subdural hematoma, epidural hematoma, subarachnoid hemorrhage, cerebral contusion and diffuse axonal injury. Patients with signs of skull fracture, scalp lacerations or soft tissue injury but with none of the above signs of internal brain injury were classified as CT-negative. Based on these criteria, 23 patients were classified as CT-positive and 20 were CT-negative (in time profile experiments). FIG. 9 displays time profiles of MBP levels, in CT-positive and CT-negative subjects, stratified by CT result. Mixed model analyses revealed that the mean fitted curve for MBP as a function of time after TBI is significantly higher for CT-positive subjects (p=0.002, likelihood ratio test). The thick solid line and thick dotted line represent fitted models for MBP vs. time from TBI for CT-positive and CT-negative subjects, respectively. FIG. 10 displays MBP levels as a function of CT result, stratified by time after injury. The separation between CT-positive and CT-negative in terms of MBP levels is greatest in the time periods of 3 to 9 hours post-TBI (p=0.0018, 3 to 4 hours; p=0.019, 4 to 6 hours; p=0.0014, 6 to 9 hours). FIG. 8 displays time profiles for S-100β, stratified by CT result. The thick dotted line represents the fitted model for S-100(3 vs. time from TBI in all subjects. FIG. 14 displays time profiles for NSE, stratified by CT result. FIGS. 8 and 14 revealed no significant differences in the mean time profiles between CT-positive and CT-negative subjects, in terms of either S-100β or NSE levels (p=0.844 for S-100β; p=0.406 for NSE).

Previous researchers have determined that S-100β is superior to NSE in predicting the outcome status of both mild and severe TBI subjects (De Kruijk et al. Acta Neurologica Scandinavica 103:175-179 2001; Ingebrigtsen et al. Neurology and Neuroscience 21:171-176 2003; Raabe et al. British Journal of Neurosurgery 13:56-59 1999). Wunderlich et al. (Stroke 30:1190-1195 1999) showed that serum S-100β levels in acute stroke patients were predictive of neurological outcome at discharge, and that serum NSE levels or lesion volumes obtained from CT scans did not add predictive value after adjusting for S-100β concentrations. Herrmann et al. (Journal of Neurology, Neurosurgery and Psychiatry 70:95-100 2001) found that the initial S-100β level obtained from TBI subjects presenting with predominantly minor head injuries predicted adverse neuropsychological outcomes after 2 weeks and after 6 months, and that S-100β was a better predictor of both short-term and long-term outcome than NSE or intracranial pathology as detected on the CT scan. Researchers have speculated that the long biological half-life and slow elimination rate of NSE render it ineffectual for distinguishing between primary and secondary brain injury (Quereshi, AI Critical Care Medicine 30:2778-2779 2002). NSE is also present in erythrocytes, and serum NSE levels are markedly affected by hemolysis, whereas S-100β levels are not (Ishida et al. Journal of Cardiothoracic and Vascular Anesthesia 17:4-9 2003).

The S-100β assay of the instant invention has a detection limit of 10 pg/mL, which is lower than that for other commercially available S-100β assays (Rothermundt et al. Microscopy Research and Technique 60:614-632 2003). Furthermore, the 98^{th} percentile reference limit for this assay in a healthy adult control population is 21 pg/mL (Takahashi et al. Clinical Chemistry 45:1307-1311 1999), whereas other commercial assays have normal reference limits exceeding 100 pg/mL (Anderson et al. Neurosurgery 48:1255-1260 2001). The differences in S-100β assay specificities would account for the fact that the serum S-100β levels observed in the instant example are generally lower than those reported in previous studied of S-100β in TBI (Rothermundt et al. Microscopy Research and Technique 60:614-632 2003).

There is evidence in the literature to suggest that MBP is released into the CSF and subsequently into the general circulation following acute neurological events. MBP is well-established as a marker of clinical activity in multiple sclerosis patients (Cohen et al. New England Journal of Medicine 295:1455-1457 1976) and has also been shown to correlate with cerebral damage in acute stroke patients (Strand et al. Stroke 15:138-144 1984). Yamazaki et al. (Surgical Neurology 43:267-271 1995) found a correlation between serum MBP levels and severity of TBI in acute head injury patients. Ng et al. performed comprehensive histological post-mortem examinations of brains of 22 victims of blunt non-penetrating head trauma and found that 17 of these cases exhibited myelin damage as detected by MBP immunostaining (Clinical Neurology and Neurosurgery 96: 24-31 1994).

The study described herein reveals that S-100β, NSE and MBP are released into the sera of TBI subjects in elevated quantities relative to non-TBI subjects, and that S-100β can serve as an aid in predicting short-term outcome status among TBI subjects. Additionally, MBP can aid in predicting the existence of brain injury as detected by CT scanning. Application of these markers in diagnostic tests will improve the efficiency and quality of care available for patients suffering from TBI, and thus potentially limit the occurrence of long-term adverse effects in these patients.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The oligonucleotides, peptides, polypeptides, antibodies, biologically related compounds, methods, procedures, techniques and diagnostic kits described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for predicting outcome for a subject suffering from traumatic brain injury (TBI) comprising the steps of;
(a) obtaining a sample of body fluid from said subject;
(b) contacting said sample of body fluid with at least one antibody that specifically binds a β subunit of S-100 protein, wherein at least one antibody is immobilized on a solid support; and
(c) determining binding of at least one antibody to said β subunit of S-100 protein in said sample of body fluid wherein a level of said β subunit of S-100 protein elevated above 39 pg/mL predicts outcome for a subject suffering from traumatic brain injury (TBI).

2. The method as in claim 1 wherein said bound antibody specifically binds the β subunit in ββ and αβ isoforms of S-100 protein.

3. The method as in claim 1 wherein said sample of body fluid is selected from the group consisting of serum, plasma, urine, lymph and cerebrospinal fluid (CSF).

4. The method as in claim 1 wherein said steps of contacting and determining are carried out by immunoassay.

5. The method as in claim 4 wherein said immunoassay is a sandwich enzyme-linked immunosorbent assay (ELISA).

6. A method for confirming the existence of brain injury in a subject by correlating a level of myelin basic protein (MBP) in a body fluid of said subject with results of a computer-assisted tomographic (CT) scan comprising the steps of;
(a) performing a CT scan on said subject;
(b) determining a result by observing pictures produced by said CT scan, wherein evidence of an abnormality in said pictures categorizes said subject as CT-positive;
(c) obtaining a sample of body fluid from said subject;
(d) contacting said sample of body fluid with at least one antibody that binds myelin basic protein (MBP), wherein at least one antibody is immobilized on a solid support; and
(e) determining binding of at least one antibody to said myelin basic protein (MBP) in said sample of body fluid wherein a level of said myelin basic protein (MBP) elevated above 76 pg/mL correlates with results of said CT scan thereby confirming the existence of brain injury in said subject.

7. The method as in claim 6 wherein said sample of body fluid is selected from the group consisting of serum, plasma, urine, lymph and cerebrospinal fluid (CSF).

8. The method as in claim 6 wherein said steps of contacting and determining of step (e) are carried out by an immunoassay.

9. The method as in claim 8 wherein said immunoassay is a sandwich enzyme-linked immunosorbent assay (ELISA).

10. A method as in claim 6 wherein said abnormality of step (b) is selected from the group consisting of subdural hematoma (SDH), epidural hematoma (EDH), subarachnoid hematoma (SAH), cerebral contusion and diffuse axonal injury (DAI).

11. A method for determining whether a subject suspected of having symptoms of traumatic brain injury (TBI) should be referred for a computer-assisted tomographic (CT) scan comprising the steps of:
(a) obtaining a sample of body fluid from said subject;
(b) contacting said sample of body fluid with at least one antibody that binds myelin basic protein (MBP), wherein at least one antibody is immobilized on a solid support; and
(c) determining binding of at least one antibody to said myelin basic protein (MBP) in said sample of body fluid wherein a level of said myelin basic protein (MBP) elevated above 76 pg/mL determines that said subject should be referred for a computer-assisted tomographic (CT) scan.

12. The method as in claim 11 wherein said sample of body fluid is selected from the group consisting of serum, plasma, urine, lymph and cerebrospinal fluid (CSF).

13. The method as in claim 11 wherein said steps of contacting and determining are carried out by an immunoassay.

14. The method as in claim 13 wherein said immunoassay is a sandwich enzyme-linked immunosorbent assay (ELISA).
